(19) United States
(12) Patent Application Publication
(10) Pub. No.: US 2026/0330231 A1
(43) Pub. Date: Oct. 22, 2026

(54) Training Multilevel Classifiers for Enhanced Clinical Data Processing, Modeling, and Analysis

(71) Applicant: Cerner Innovation, Inc., Kansas City, MO (US)

(72) Inventors: Natalee Agassi, Blue Bell, PA (US); Amitabh Saikia, Mountain View, CA (US); Jigar Shirish Patel, Lenexa, KS (US); Raman Grover, Palo Alto, CA (US); Vadim Khotilovich, Leawood, KS (US); Raefer Christopher Gabriel, San Mateo, CA (US)

(73) Assignee: Cerner Innovation, Inc., Kansas City, MO (US)

(21) Appl. No.: 19/572,212

(22) Filed: Mar. 19, 2026

Related U.S. Application Data
(60) Provisional application No. 63/792,124, filed on Apr. 21, 2025.

(51) Int. Cl. G06N 3/086; G16H 50/70

(52) U.S. Cl. CPC G06N 3/086; G16H 50/70

(57) ABSTRACT

Techniques for training a multilevel classifier for assisting in clinical data processing, modeling, and analysis are disclosed. The system uses artificial intelligence (AI) to identify previously unidentified relationships between clinical expressions and genomic expressions. The clinical expressions, genomic expressions, and the newly identified relationships between the clinical expressions and genomic expressions are used by the system to train a multilevel classifier. The relationships identified by AI identify a patient condition within the hierarchical structure, thereby aiding in medical modeling and analysis. The relationships may identify treatments for the patient condition, aid in early disease detection, and/or provide other insights for use by clinicians.

### INCORPORATION BY REFERENCE; DISCLAIMER

Each of the following applications and any parent patent applications (provisionals, non-provisionals, international, and foreign) to which this application claims priority to, directly or indirectly, are hereby incorporated by reference in their entirety to the same extent as if fully and explicitly recited herein. Any incorporation by reference is limited such that no subject matter is incorporated that is contrary to the explicit disclosure herein. The applications being incorporated by reference include at least: U.S. Application No. 63/792,124 filed on Apr. 21, 2025.

The Applicant hereby rescinds any disclaimer of claim scope in the parent application(s) or the prosecution history thereof and advises the USPTO that the claims in this application may be broader than any claim in the parent application(s).

### TECHNICAL FIELD

The present disclosure relates to computer-implemented systems and methods for processing, modeling, and analyzing complex healthcare-related information. In particular, the present disclosure relates to computing architectures, data processing pipelines, and machine-learning-enabled systems for handling heterogeneous clinical, biomedical, and patient-related data within electronic computing environments.

### BACKGROUND

Healthcare delivery increasingly relies on computerized systems to store, manage, and process medical and biomedical information. Such systems are commonly used across clinical, administrative, and research settings to support various activities, including patient care documentation, clinical analysis, population health management, and biomedical research. Medical data handled by these systems may include, for example, clinical observations, diagnostic information, laboratory results, genomic data, and other forms of patient-related information.

To facilitate interoperability and consistent interpretation, healthcare information is often represented using standardized terminologies, data models, and structured formats. These representations enable computerized systems to exchange, aggregate, and analyze data across different platforms, applications, and organizations. In addition to structured data, healthcare systems frequently manage unstructured or semi-structured data, such as narrative text, reports, or externally sourced biomedical information.

Healthcare information systems often include multiple interacting components, such as data repositories, processing engines, analytics services, and user interfaces. These components may be configured to exchange data through standardized interfaces and protocols as well as support a variety of workflows involving data retrieval, transformation, and computational analysis. Such systems are designed to accommodate ongoing updates to data, models, and computational logic while maintaining operational continuity.

The approaches described in this section are approaches that could be pursued but not necessarily approaches that have been previously conceived or pursued. Therefore, unless otherwise indicated, it should not be assumed that any of the approaches described in this section qualify as prior art merely by virtue of their inclusion in this section.

### BRIEF DESCRIPTION OF THE DRAWINGS

The embodiments are illustrated by way of example and not by way of limitation in the figures of the accompanying drawings. It should be noted that references to “an” or “one” embodiment in this disclosure are not necessarily to the same embodiment, and they mean at least one. In the drawings:

FIG. 1 illustrates a system in accordance with one or more embodiments;

FIG. 2 illustrates an example set of operations for training a multilevel classifier for assisting in clinical data processing, modeling, and analysis in accordance with one or more embodiments;

FIG. 3 illustrates an example system for training a multilevel classifier;

FIG. 4A illustrates a machine learning (ML) engine in accordance with one or more embodiments;

FIG. 4B illustrates an example set of operations of an ML engine in accordance with one or more embodiments; and

FIG. 5 shows a block diagram that illustrates a computer system in accordance with one or more embodiments.

### DETAILED DESCRIPTION

In the following description, for the purposes of explanation, numerous specific details are set forth to provide a thorough understanding. One or more embodiments may be practiced without these specific details. Features described in one embodiment may be combined with features described in a different embodiment. In some examples, well-known structures and devices are described with reference to a block diagram form to avoid unnecessarily obscuring the present disclosure.
1. GENERAL OVERVIEW
2. CLINICAL DATA PROCESSING AND MODELING SYSTEM ARCHITECTURE
3. TRAINING A MULTILEVEL CLASSIFIER FOR SUPPORTING CLINICAL DATA PROCESSING, MODELING, AND ANALYSIS
4. EXAMPLE SYSTEM FOR TRAINING A MULTILEVEL CLASSIFIER
5. MACHINE LEARNING ARCHITECTURE
6. GENERATIVE MODELS
7. PRACTICAL APPLICATIONS, ADVANTAGES, AND IMPROVEMENTS
8. HARDWARE OVERVIEW
9. MISCELLANEOUS; EXTENSIONS

### 1. General Overview

One or more embodiments train a multilevel classifier within a computer-implemented system configured for processing healthcare-related data. The system employs AI techniques to analyze clinical expressions and genomic expressions and computationally derive relationships between such expressions based on patterns present in training data. The clinical expressions, genomic expressions, and the derived relationships are represented as machine-readable data structures and provided as inputs for training the multilevel classifier. The trained multilevel classifier is configured to operate over hierarchical data representations and to produce structured outputs corresponding to classifications within the hierarchy.

One or more embodiments generate vector embeddings corresponding to clinical expressions, relationships among clinical expressions, and clinical concepts with associated attributes. The system further generates vector embeddings corresponding to genomic expressions. Using the respective vector embeddings, the system performs similarity and association computations to determine relationships between genomic concepts and clinical concepts and generates vector embeddings representing the determined relationships. The system classifies the vector embeddings, including the relationship embeddings, and uses the classified vector embeddings as training data for the multilevel classifier.

One or more embodiments described in this Specification and/or recited in the claims may not be included in this General Overview section.

### 2. Clinical Data Processing and Modeling System Architecture

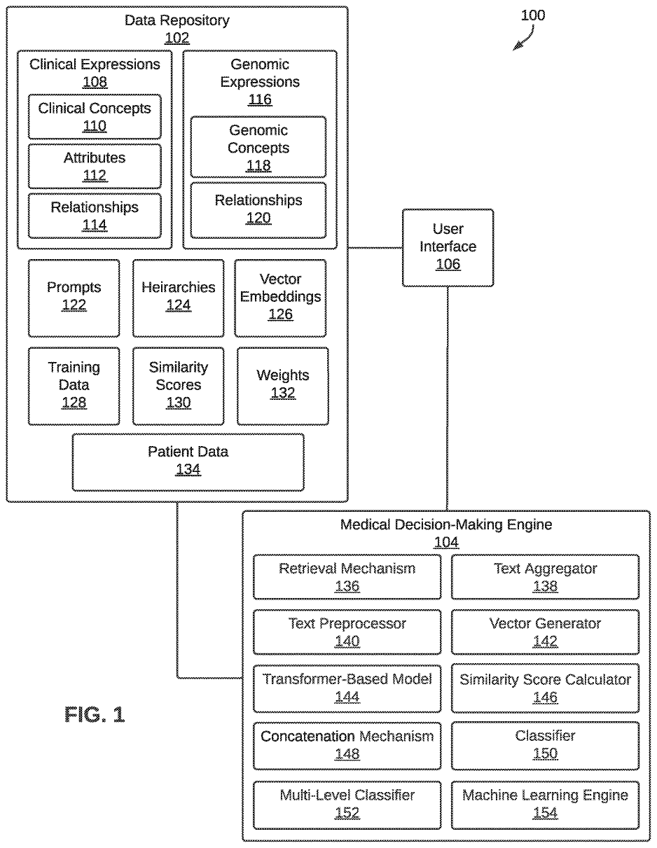

FIG. 1 illustrates a system 100 in accordance with one or more embodiments. As illustrated in FIG. 1, system 100 includes a data repository 102, a clinical data processing and modeling engine 104. In one or more embodiments, the system 100 may include more or fewer components than the components illustrated in FIG. 1. The components illustrated in FIG. 1 may be local to or remote from each other. The components illustrated in FIG. 1 may be implemented in software and/or hardware. Each component may be distributed over multiple applications and/or machines. Multiple components may be combined into one application and/or machine. Operations described with respect to one component may instead be performed by another component.

In one or more embodiments, data repository 102 is any type of storage unit and/or device (e.g., a file system, database, collection of tables, or any other storage mechanism) for storing data. Furthermore, data repository 102 may include multiple different storage units and/or devices. The multiple different storage units and/or devices may or may not be of the same type or located at the same physical site. Furthermore, data repository 102 may be implemented or executed on the same computing system as clinical data processing and modeling engine 104. Additionally, or alternatively, data repository 102 may be implemented or executed on a computing system separate from clinical data processing and modeling engine 104. Data repository 102 may be communicatively coupled with clinical data processing and modeling engine 104 via a direct connection or via a network.

Information describing training a multilevel classifier for clinical data processing and modeling may be implemented across any of components within the system 100. However, this information is illustrated within the data repository 102 for purposes of clarity and explanation.

In one or more embodiments, data repository 102 includes clinical expressions 108. Clinical expressions 108 refer to a standardized way of recording and communicating clinical information using specific terminologies, ontologies, and other structured data formats. Clinical expressions 108 maintain clarity, accuracy, and consistency in medical records, facilitating communication among healthcare providers and enabling advanced data processing, modeling, and analysis systems.

Various standards and frameworks are used to create and manage clinical expressions, ensuring the clinical expressions are precise, interoperable, and usable across different health information systems. Examples of standards for clinical expressions include Systematized Nomenclature of Medicine-Clinical Terms (SNOMED CT), Logical Observation Identifiers Names and Codes (LOINC), and International Classification of Diseases (ICD). SNOMED CT is a comprehensive, multilingual clinical healthcare terminology, that allows for the systematic encoding of medical conditions and procedures. SNOMED CT supports both pre-coordinated and post-coordinated expressions. LOINC is primarily used for lab tests and clinical observations. LOINC is used to standardize the representation of test results and other measurements. ICD is used globally for morbidity and mortality statistics. ICD codes diseases and a wide variety of signs, symptoms, abnormal findings, and external causes of injury or diseases.

In one or more embodiments, clinical expressions 108 include a combination of standardized terminologies and codes that describe diseases, symptoms, procedures, medications, and other relevant clinical facts. Clinical expressions 108 can be simple, representing a single concept, i.e., pre-coordinated expressions, or complex, involving multiple concepts to convey detailed clinical scenarios, i.e., post-coordinated expressions. Pre-coordinated expressions are predefined terms within a clinical terminology system that describe a single clinical concept or a simple combination of clinical concepts. For example, “type 2 diabetes mellitus” is a pre-coordinated expression in systems like SNOMED CT, where the expression represents a specific disease without needing further modification. Post-coordinated expressions are constructed by combining multiple clinical concepts to describe more complex situations. For instance, describing a patient who has “type 2 diabetes mellitus with neuropathy” might involve linking the clinical concept of “type 2 diabetes mellitus” with another clinical concept like “neuropathy” using specific relational terms defined within a terminology system like SNOMED CT.

In one or more embodiments, clinical expressions 108 include clinical concepts 110. Clinical concepts 110 are primary building blocks within any healthcare terminology system. Clinical concepts 110 represent discrete pieces of medical knowledge, including diseases, symptoms, procedures, medications, and devices. Each clinical concept is assigned a unique identifier that can be universally recognized across different systems and software, ensuring consistency and clarity. For example, “Asthma” is a clinical concept representing a chronic inflammatory disease of the airways.

In one or more embodiments, clinical expressions 108 include attributes 112. Attributes 112 are properties or characteristics that can be used to define or modify clinical concepts. Attributes 112 provide additional specificity and detail, allowing clinical expressions to convey more precise information about a patient's condition or treatment. Attributes 112 are crucial for tailoring a generic concept to a specific clinical situation. In an example, for the clinical concept “fracture of femur,” attributes could include: severity—severe, moderate, or minor; location—distal, proximal, or midshaft; and status—open or closed. Attributes refine a clinical concept, enabling clinicians to describe a patient's condition more accurately.

In one or more embodiments, clinical expressions 108 include relationships 114. Relationships 114 connect clinical concepts to one another or to their attributes, forming structured and meaningful clinical expressions. Relationships 114 can be hierarchical, e.g., “is-a” or associative, e.g., “has-property,” “part-of,” or “caused-by,” and are essential for building complex medical knowledge structures within clinical terminologies. Hierarchical relationships define generalization-specialization relationships between concepts. For example, “Type 1 diabetes mellitus” is a type of “Diabetes mellitus.” Associative relationships include “has-finding site,” e.g., “malignant neoplasm of the breast” has a finding site of “Breast structure”; “associated morphology,” e.g., “Adenocarcinoma” has associated morphology of “Adenoma”; and “causative agent, e.g., “strep throat” caused by “Streptococcus.”

In one or more embodiments, data repository 102 includes genomic expressions 116. Genomic expressions 116 refer to the representation and annotation of genomic data using standardized vocabularies and structure formats. Genomic expressions 116 ensure that genomic information is communicated effectively, interoperable across different systems, and analyzable in a clinical setting. The use of standardized formats facilitates the integration of genomic data into electronic health records (EHRs), enhances modeling and analysis support systems, and supports robust genetic research.

Standardized vocabularies and formats for genomic expressions include Human Genome Variation Society (HGVS) Nomenclature, LOINC, Gene Ontology (GO) and SNOMED CT. HGVS Nomenclature standardizes the way genetic variants are described. For example, the notation “BRCA1c.5266dupC” precisely specifies a particular duplication mutation in the BRCA1 gene. LOINC are used for specific genetic tests, ensuring that test results are communicated in a universally understood format across different laboratories and EHR systems. GO provides a structured vocabulary that can be used to describe the roles of genes and gene products across species. GO terms describe gene functions, biological processes, and cellular components, such as “GO:0008150” for biological processes. SNOMED CT can be used to annotate and categorize clinical phenotypes associated with genetic conditions.

The Unified Medical Language System (UMLS) is a comprehensive set of resources and tools developed by the National Library of Medicine (NLM) that brings together many health and biomedical vocabularies and standards to enable interoperability between computer systems. UMLS integrates and connects terms from various established vocabularies and ontologies. GO terms are part of UMLS. The National Center for Biotechnology Information (NCBI) Taxonomy is another source within UMLS which provides information on the relationships between biological organisms that can be relevant to understanding certain genomic processes. HUGO Gene Nomenclature Committee (HGNC) provides UMLS with specific detailed genomic expressions and authoritative names for human genes. This includes gene symbols and names which are crucial for the precise identification and discussion of genomic studies.

In one or more embodiments, genomic expressions 116 include genomic concepts 118. Genomic concepts 118 relate to the genetic information encoded in DNA, including genomes, genes, gene variants, and their functions and interactions within an organism. Genes and gene variants are the basic units of heredity and include variations that may influence health, disease susceptibility, and responses to drugs (pharmacogenomics). Gene expression refers to the process by which the instructions in DNA are converted into a functional product, such as a protein. This concept also includes how various factors (environmental, developmental, pathological) can affect the expression levels of genes. Epigenetics involves changes in gene activity and expression that do not involve alterations to the genetic code but are heritable and potentially reversible. These changes can result from external or environmental factors. Genomic signatures refer to patterns in the genome, e.g., mutations or gene expression changes, that can be associated with specific diseases, conditions, or responses to treatment.

In one or more embodiments, genomic expressions 116 include relationships 120. Relationships 120 explain how different genetic components interact within the genome and with external factors, affecting biological functions and phenotypes. Relationships include gene-gene interactions, gene-environment interactions, genotype-phenotype relationships, regulatory networks, and pathway involvement. Gene-gene interactions (epistasis) refers to the effect of one gene on the function or expression of another gene. The outcome of such interactions can influence an organism's phenotype in ways that are not predictable by simply considering the effects of each gene separately. For example, the interaction between genes responsible for pigment production can result in variations in coloration that are not explained by the action of any single gene. Gene-environment interactions occur when the environment affects gene expression, influencing an individual's phenotype. For example, nutritional factors can affect gene expression related to metabolism and growth, such as how dietary folate can influence gene expression linked to neural tube defects. Genotype-phenotype relationships are direct relationships between the genetic makeup (genotype) of an organism and its observable characteristics (phenotype). For example, specific mutations in the BRCA1 or BRCA2 genes have a strong relationship with an increased risk of breast and ovarian cancers. Genes are often part of complex regulatory networks that control gene expression through feedback loops and signaling cascades. For example, regulation of cell cycle genes through networks involves both promoters and inhibitors of cell division. Genes often function as part of broader biological pathways that govern cellular processes such as metabolism, signal transduction, or DNA repair. For example, the p53 gene plays a critical role in the DNA damage response pathway, helping to prevent cancer by repairing DNA or inducing apoptosis in cells with irreparable damage.

In one or more embodiments, data repository 102 includes prompts 122. Prompts 122 are pieces of text input into a model to initiate a response or output. Prompts 122 set the context or task for the model. Prompts 122 are used to “prime” the model on what the model needs to do. The effectiveness of a prompt can significantly affect the quality and relevance of the model's output. Depending on the model and the task, prompts can range from simple questions or statements to complex paragraphs. The prompt may include specific instructions, context information, or styled text to guide the desired output.

In one or more embodiments, data repository 102 includes hierarchies 124. Hierarchies 124 refer to fundamental organizational structures that arrange elements or entities in a system of ordered levels, with each level subordinate to the one above it. Hierarchies 124 are used to organize clinical concepts. For example, in SNOMED CT, diseases are categorized under broader categories, which then can be broken down into more specific conditions. This hierarchical categorization includes various attributes and relationships, making it easier to find and use clinical data systematically. ICD uses a hierarchical system to categorize diseases and health conditions. Hierarchies 124 may be used to organize genomic concepts. For example, GO is a major bioinformatics initiative that provides a controlled vocabulary to describe the roles of genes and gene products across species in a hierarchical format. This ontology covers three domains: cellular component, molecular function, and biological process, each structured hierarchically from broad to specific terms. Genetic variants can be categorized into a hierarchy based on their impact on protein function, from benign variants to those that cause disease. This hierarchical classification aids in assessing the clinical significance of genetic findings.

In one or more embodiments, data repository 102 includes vector embeddings 126. Vector embeddings 126 refer to text that has been converted to a numeric format. The vector embeddings 126 are representations of individual words for text analysis, typically in the form of a real-valued vector. Vector embeddings 126 may represent individual text items or may represent an aggregation of text items. As will be described in further detail below with respect to clinical data processing and modeling engine 104, vector embeddings 126 may be formed using various word embedding techniques. Vector embeddings 126 represent (i) pre-coordinated and post-coordinated expressions, (ii) clinical concepts with associated attributes, (iii) concepts with hierarchical relationships, (iv) genomic expressions, and (v) relationships between clinical concepts and genomic concepts.

In one or more embodiments, data repository 102 includes training data 128. Training data 128 refers to datasets used to train machine learning (ML) models. Training data 128 may include a set of input-output pairs. For supervised learning models, both the inputs, i.e., features, and the outputs, i.e., labels or targets, are specified. Inputs can be any form of data such as numbers, text, images, etc., and outputs are generally categories (in classification) or continuous values (in regression). Training data 128 may include (i) annotated clinical texts linked to clinical expressions, (ii) genomic datasets, e.g., BRCA1 mutation associations, and/or (iii) structured knowledge bases, e.g., RxNorm, UMLS, ICD-10, SNOMED CT, PubMed, MEDLINE biomedical corpora.

In some embodiments, data repository 102 includes similarity scores or values 130. Similarity scores 130 provide an indication of the similarity between vector embeddings 126. The higher the similarity scores 130, for example, the closer to 1.0, depending on the scale, the greater a semantic match between vector embeddings 126, e.g., between a vector embedding for a clinical expression and a vector embedding for a genomic expression. Similarity scores 130 may be weighted to reflect the relevance of the type of data used to calculate vector embeddings 126.

In one or more embodiments, data repository 102 includes weights 132. Weights 132 refer to parameters within machine learning models that adjust during training to minimize the difference between the actual output of the model and the expected output, i.e., the target. In neural networks, each neuron's output is computed as a weighted sum of its inputs, which is then passed through a non-linear function, i.e., activation function. Weights 132 determine the strength and sign of the input signals in computing the output.

In one or more embodiments, data repository 102 includes patient data 134. Patient data 134 refers to a broad range of information collected in medical settings that pertains to individual patients. Patient data may include demographic information, medical history, medication information, laboratory and test results, clinical notes, genetic information, and/or lifestyle information. Demographic information includes basic information about the patient such as name, age, gender, date of birth, ethnicity, and contact details. Medical history includes detailed records of a patient's past medical conditions, surgeries, hospitalizations, family medical history, allergies, and chronic diseases. Medication information includes information about medications a patient is taking or has taken, including dosages, frequency, and any adverse reactions. Laboratory and test results include results from blood tests, biopsies, imaging tests (like X-rays, MRIs), and other diagnostic tests. Clinical notes include notes taken by doctors, nurses, and other healthcare providers during patient visits. Genetic information includes data related to a patient's genetic markers that can indicate predisposition to certain health conditions or diseases. Lifestyle information include information about a patient's lifestyle choices, such as diet, exercise habits, tobacco and alcohol use, and other behaviors that significantly impact health.

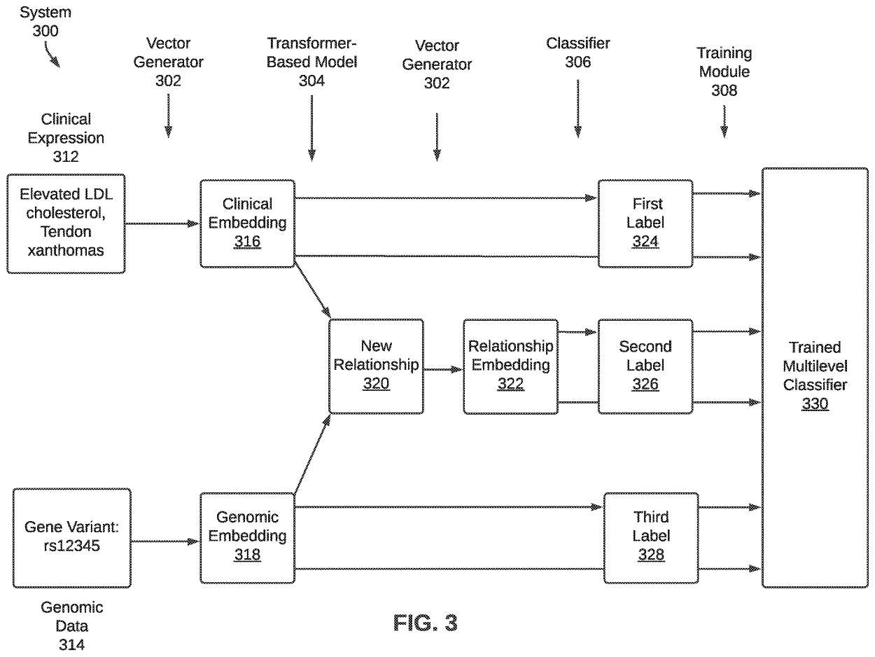

In one or more embodiments, clinical data processing and modeling engine 104 refers to hardware and/or software configured to perform operations described herein for training a multilevel classifier to support clinical data processing and modeling. Examples of operations for training a multilevel classifier to support clinical data processing and modeling are described below with reference to FIG. 3.

In an embodiment, clinical data processing and modeling engine 104 is implemented on one or more digital devices. The term “digital device” generally refers to any hardware device that includes a processor. A digital device may refer to a physical device executing an application or a virtual machine. Examples of digital devices include a computer, a tablet, a laptop, a desktop, a netbook, a server, a web server, a network policy server, a proxy server, a generic machine, a function-specific hardware device, a hardware router, a hardware switch, a hardware firewall, a hardware network address translator (NAT), a hardware load balancer, a mainframe, a television, a content receiver, a set-top box, a printer, a mobile handset, a smartphone, a personal digital assistant (PDA), a wireless receiver and/or transmitter, a base station, a communication management device, a router, a switch, a controller, an access point, and/or a client device.

In one or more embodiments, clinical data processing and modeling engine 104 includes a retrieval mechanism 136. Retrieval mechanism 136 refers to hardware and/or software configured to perform operations described herein for accessing and retrieving information from data sources. This may include database retrieval and information retrieval. In relational databases, SQL (Structured Query Language) is used for querying and retrieving data. SQL allows for specifying exactly what data to retrieve and how the data should be processed. In NoSQL databases, retrieval mechanisms can vary significantly depending on the type of NoSQL database, e.g., document, key-value, graph, or column store. These might use simpler, more scalable retrieval methods suited to the specific data model. Information retrieval is used primarily in search engines and large content management systems designed to handle unstructured data like text. Techniques include keyword search, semantic search, and full-text search. Retrieval mechanism 136 may use application programming interfaces (APIs) to access data. Retrieval mechanism 136 may employ web scrapping techniques to acquire data from websites.

In one or more embodiments, clinical data processing and modeling engine 104 includes text aggregator 138. Text aggregator 138 refers to hardware and/or software configured to perform operations described herein for collecting, compiling, and/or synthesizing textual content from various sources. Text aggregator 138 may combine or concatenate data from various fields in preparation for vector embedding operation.

In one or more embodiments, clinical data processing and modeling engine 104 includes text preprocessor 140. Text preprocessor 140 refers to hardware and/or software configured to perform operations described herein for preparing text prior to generating vector embeddings. Operations may include converting the text into lowercase, removing white spaces, prefix removal, punctuation removal, and/or retaining numeric tokens. Text is converted to lowercase to provide uniformity to the text. Prefix removal includes removing prefixes such as “z,” “zz,” “zzz.” Punctuation removal is performed to remove any non-alphanumeric characters. Text preprocessing may include handling special characters, removing unwanted text, and custom preprocessing. Handling special characters includes addressing symbols and special characters. Custom preprocessing includes attending to consumer specific text, such as synonyms, abbreviations, and shorthand.

In one or more embodiments, clinical data processing and modeling engine 104 includes vector generator 142. Vector generator 142 refers to hardware and/or software configured to perform operations described herein for performing one or more vector embedding functions. Vector embedding functions are mathematical functions that map objects, such as words, sentences, or other data points, into vector representations in a multi-dimensional space. These vector representations are used to capture the semantic or contextual meaning of the objects in a numerical format that can be easily processed by machine learning algorithms.

In some embodiments, the vector embedding functions are word embedding techniques. Word embedding techniques use natural language processing (NLP) and machine learning to represent words as dense vectors of real numbers. Word embedding techniques aim to capture the semantic and syntactic meaning of words as well as their relationships with other words in a language. Word embedding techniques include Term Frequency-Inverse Document Frequency (TF-IDF), Word2Vec, Global Vectors (GloVe), BioWordVec FastText, and Bidirectional Encoder Representation (BERT).

In one or more embodiments, word embedding techniques include Clinical-BERT or Self-Alignment Pretraining for Biomedical Entity Representations (SAPBERT). SAPBERT is a specialized Large Language Model (LLM) that is a pre-trained BERT model trained on Medical Entity Linking (MEL) tasks. MEL maps various entities to unified concepts in the medical knowledge graph. Word representation learning faces a significant challenge due to the existence of heterogeneous names. For example, in healthcare, terms like ‘nostril’ and ‘nare’ are used interchangeably but yield considerably different embedding representations when generated by models not specifically trained for MEL. SAPBERT works on self-alignment of biomedical entity representation such that the semantically similar entities belonging to the same concept are brought closer in the embedding space, thus forming compact clusters. SAPBERT leverages UMLS, the largest collection of biomedical concepts and synonyms and collates the synonyms from various controlled vocabularies, e.g., SNOMED CT, MeSH, Gene Ontology, RxNorm, and OMIM. SAPBERT performs better compared to other variants of BERT like BioBERT, Clinical-BERT with respect to the MEL challenges. The SAPBERT model can accurately capture fine-grained semantic relationships and heterogeneous naming in the biomedical domain compared to other variants of BERT. The ability of SAPBERT to handle out-of-vocabulary terms, misspelled words, and rare medical terms provides a significant advantage over other models.

In one or more embodiments, clinical data processing and modeling engine 104 includes transformer-based model 144. Transformer-based model 144 refers to hardware and/or software configured to perform operations described herein for identifying relationships between vector embeddings of clinical expressions and genomic expressions. Self-attention mechanisms inherent in transformers allows the transformer-based model to dynamically weigh the importance of different parts of input data relative to each other, making the transformer-based model 144 adept at uncovering subtle and intricate relationships. Transformer-based model 144 may include BioBERT, Enhanced Representation through kNowledge Integration (ERNIE), ClinicalBERT, or PubMedBERT.

In one or more embodiments, clinical data processing and modeling engine 104 includes a similarity score calculator 146. Similarity score calculator 146 refers to hardware and/or software configured to perform operations described herein for calculating similarity between vector embeddings for clinical expressions and vector embeddings for genomic expressions. Similarity matching or similarity retrieval can be used to find relationships between clinical expression and genomic expressions. Similarity matching measures the similarity between a clinical expression and a genomic expression, based on certain features or characteristics, i.e., attributes. To measure similarity, a distance measure or similarity metric is chosen. Common distance measures include Euclidean distance, cosine similarity, and Jaccard similarity. When dealing with large datasets, an index may be created to speed up the search process. An index is a data structure that organizes the data in a way that allows for efficient retrieval of similar items.

In one or more embodiments, clinical data processing and modeling engine 104 includes concatenation mechanism 148. Concatenation mechanism 148 refers to hardware and/or software configured to perform operations described herein for merging two or more data elements together. Concatenation mechanism 148 may combine two or more sequences or sets of items end-to-end to form a single sequence or set.

In one or more embodiments, clinical data processing and modeling engine 104 includes classifier 150. Classifier 150 refers to hardware and/or software configured to perform operations described herein for classifying vector embeddings 126. Classifier 150 is a type of algorithm or model trained to distinguish between different classes or categories based on input data. The primary function of classifier 150 is to assign a category or label to new, unseen data based on the patterns learned from training data.

In one or more embodiments, classifier 150 is a multi-output classifier. Multi-output classifiers are a type of machine learning model that is capable of predicting multiple dependent or independent output variables from a single set of input features. Multi-output classifiers are an extension of traditional single-output classifiers and handle the complexity of multiple prediction tasks in an integrated way. Unlike standard classification models that predict a single label or continuous value per instance, multi-output classifiers predict several outputs at once. The outputs can be a mix of categorical. Multi-output classifiers can be designed to exploit the potential correlations among outputs. For example, in a scenario predicting various diseases a patient might have, the presence of one disease might influence the likelihood of another. By modeling multiple tasks together, multi-output classifiers can be more efficient than separate models for each output, especially when there are shared underlying patterns across outputs.

In one or more embodiments, clinical data processing and modeling engine 104 includes multilevel classifier 152. Multilevel classifier 152, also referred to as a hierarchical classifier, refers to hardware and/or software configured to perform operations described herein for handling problems where classes are arranged in a hierarchical structure. Classes in multilevel classification are organized in a hierarchy that can be visualized as a tree. Each node in the tree represents a class, and child nodes represent more specific categories of their parent node. This structure allows the classifier to exploit relationships between broader and more specific classes.

In one or more embodiments, multilevel classifier 152 may include neural networks, support vector machine (SVM), or random forest. Neural networks are inspired by the structure and function of the human brain. Neural networks consist of layers of interconnected nodes or neurons, where each connection represents a weight that is adjusted during training. SVMs are based on the concept of finding a hyperplane that best divides a dataset into classes, maximizing the margin between different classes'support vectors, i.e., critical data points. Random Forest is an ensemble learning technique that builds multiple decision trees and merges them together to get a more accurate and stable prediction.

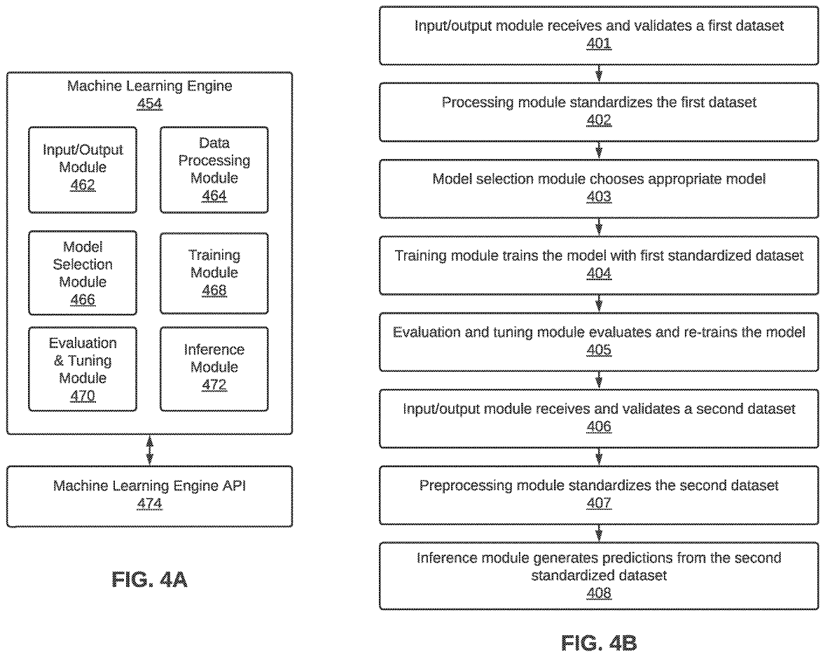

In one or more embodiments, clinical data processing and modeling engine 104 includes machine learning engine 154. Machine learning engine 154 refers to hardware and/or software configured to perform the operations described herein for training and applying machine learning models. The structure and function of machine learning engine 154 will be described below in detail with reference to FIGS. 4A and 4B.

In one or more embodiments, user interface 106 refers to hardware and/or software configured to facilitate communications between a user and clinical data processing and modeling engine 104. User interface 106 renders user interface elements and receives input via user interface elements. Examples of interfaces include a graphical user interface (GUI), a command line interface (CLI), a haptic interface, and a voice command interface. Examples of user interface elements include checkboxes, radio buttons, dropdown lists, list boxes, buttons, toggles, text fields, date and time selectors, command lines, sliders, pages, and forms.

In an embodiment, different components of user interface 106 are specified in different languages. The behavior of user interface elements is specified in a dynamic programming language such as JavaScript. The content of user interface elements is specified in a markup language, such as hypertext markup language (HTML) or XML User Interface Language (XUL). The layout of user interface elements is specified in a style sheet language such as Cascading Style Sheets (CSS). Alternatively, user interface 106 is specified in one or more other languages, such as Java, C, or C++.

### 3. Training a Multilevel Classifier for Supporting Clinical Data Processing, Modeling, and Analysis

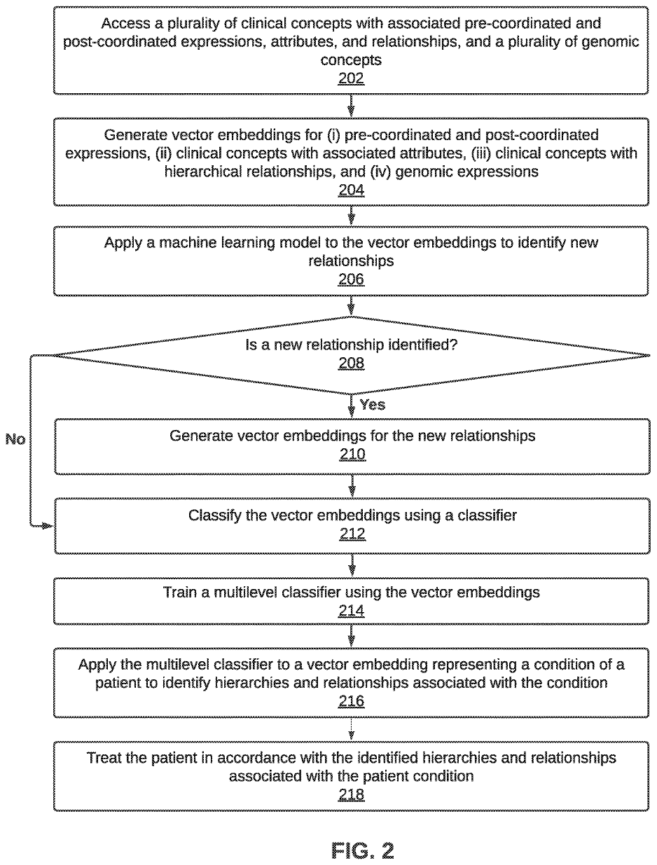

FIG. 2 illustrates an example set of operations for training a multilevel classifier for supporting clinical data processing, modeling, and analysis in accordance with one or more embodiments. One or more operations illustrated in FIG. 2 may be modified, rearranged, or omitted. Accordingly, the particular sequence of operations illustrated in FIG. 2 should not be construed as limiting the scope of one or more embodiments.

One or more embodiments access a plurality of clinical concepts with associated pre-coordinated and post-coordinated expressions, attributes, and relationships as well as a plurality of genomic concepts (Operation 202). The clinical concepts and genomic concepts may be defined according to one or more standardized terminologies or data models, including those that provide formally defined ontologies and hierarchical structures. For example, standardized clinical vocabularies, such as SNOMED CT and LOINC, provide machine-readable representations of concepts, descriptions, and inter-concept relationships.

In one or more embodiments, the ontologies and vocabularies are distributed in one or more structured data formats. Structured data formats may include Release Format 2 (RF2), Comma-Separated Values (CSV), JavaScript Object Notation (JSON), Web Ontology Language (OWL), or Resource Description Framework (RDF). These data files may encode concept identifiers, textual descriptions, attributes, hierarchical relationships, associative relationships, and/or metadata defining constraints and/or contextual usage. The system may access the plurality of clinical concepts and associated expressions as well as the plurality of genomic concepts by downloading one or more ontology or terminology distribution files from publicly available or licensed repositories associated with the respective standards.

In one or more embodiments, the system accesses the clinical concepts and genomic concepts via one or more APIs exposed by terminology services, ontology servers, and/or data providers. APIs may support query-based retrieval of concepts, expressions, attributes, and/or relationships. APIs may return results in structured formats suitable for automated processing.

In one or more embodiments, once accessed, the system loads the clinical concept data and genomic concept data into one or more structured data stores, e.g., relational databases, graph databases, document stores, or in-memory data structures. The system may normalize, index, or otherwise organize the data to support efficient querying and traversal of concept hierarchies and relationships. The system may store identifiers, descriptions, attributes, and/or relationship metadata as separate but linked records to facilitate scalable access and update operations.

In one or more embodiments, the system executes one or more queries over the structured data to generate textual or symbolic representations that correspond to clinical expressions, genomic expressions, concept attributes, and/or relationships. These representations may include, for example, concatenated descriptions, normalized text strings, and/or serialized structures that capture relevant semantic content for downstream computational processing. The generated representations may be stored, cached, and/or provided as inputs to subsequent processing stages described herein.

One or more embodiments generate vector embeddings for i) pre-coordinated and post-coordinated expressions, ii) clinical concepts with associated attributes, iii) clinical concepts with hierarchical relationships, and iv) genomic expressions (Operation 204). For each category, the system may aggregate text or symbolic representations corresponding to the respective expressions, concepts, attributes, and/or relationships. For example, aggregation may include concatenating preferred terms, synonyms, attribute values, relationship labels, hierarchical path descriptors, or other metadata associated with a given concept or expression into a unified textual or structured representation.

In one or more embodiments, prior to generating vector embeddings, the system applies one or more preprocessing operations to the aggregated text for each category. The preprocessing operations may include converting text to a normalized case, tokenizing text into words, sub words, or symbols, retaining or normalizing numeric tokens, handling punctuation and special characters, removing stop words or unwanted tokens, and/or applying domain-specific preprocessing rules. The system may apply different preprocessing pipelines to different categories of text to accommodate variations in structure, vocabulary, and/or semantic density.

Following preprocessing, the system may apply one or more vector embedding functions to the aggregated and preprocessed text to generate dense numerical vector representations. Each vector embedding encodes semantic or structural information associated with a corresponding expression, concept, and/or relationship in a format suitable for computational comparison and analysis. The system may use word-level or phrase-level embedding models to convert tokens or sequences of tokens into fixed-length vectors. The system may apply embedding models configured for longer sequences or contextual representations to capture relationships among tokens within the aggregated text.

For genomic expressions, the system may generate vector embeddings using techniques configured to operate on biological sequence data. These techniques may include k-mer-based representations, sequence segmentation, and/or embedding models trained to capture patterns within nucleotide or amino acid sequences. The system may generate embeddings independently of, or in coordination with, the embeddings generated for clinical expressions and concepts.

In one or more embodiments involving hierarchical or graph-based clinical concept structures, the system generates graph-based embeddings that preserve topological or relational properties of the underlying ontology or concept graph. These embeddings may encode parent-child relationships, sibling relationships, and/or other structural associations between concepts. In some implementations, these graph-based embeddings are generated separately from text-based embeddings and subsequently combined, concatenated, or otherwise associated with corresponding concept vectors.

The system may employ the same vector embedding function across multiple categories of text or may select different embedding functions based on the characteristics of the input data. For example, one embedding function may be selected for free-text clinical expressions, whereas another embedding function is selected for structured attributes, hierarchical descriptors, or genomic sequences. Selection of the embedding function may be based on various factors, such as input length, vocabulary size, structural complexity, or desired embedding dimensionality. The resulting vector embeddings may be stored, indexed, and/or passed to subsequent processing stages for further computational operations.

One or more embodiments apply a machine learning (ML) model to the vector embeddings to identify new relationships (Operation 206). The system trains the ML model to operate on vector embeddings corresponding to clinical concepts, genomic concepts, and associated expressions to generate machine-readable representations of relationships among such embeddings. When relationship labels or annotations are available, the system may train a supervised ML model using labeled pairs or sets of vector embeddings to predict the presence, category, or attributes of relationships. When labeled data is not available, the system may apply unsupervised or weakly supervised techniques, such as clustering algorithms, association discovery, or embedding-space proximity analysis, to identify groupings or associations based on statistical properties of the embeddings. The ML model may further be trained using predefined hierarchies or ontology-derived constraints to guide relationship generation within structured concept spaces.

In one or more embodiments, the system provides structured inputs to the ML model to cause generation of relationship data from the vector embeddings. Such inputs may include similarity-based queries executed over the embedding space, for example, by computing cosine similarity, Euclidean distance, or other vector similarity measures between selected embeddings. In addition, the system may incorporate graph-based context derived from ontology structures, such as hierarchical or associative links defined in standards including SNOMED CT, to provide additional structural signals to the ML model. These signals may be used to constrain, weight, or contextualize relationship generation operations performed by the model.

In one or more embodiments, the system employs predefined, template-based prompting structures to provide structured inputs to the ML model. A prompt template may define a parameterized query format that references one or more placeholders corresponding to concept identifiers, attributes, or embedding references. For example, a prompt template may specify a request to enumerate associations linked to a particular genomic feature identifier where the input data includes vector embeddings derived from genomic expression profiles. In another example, a prompt template may specify identification of co-occurring or interacting concepts across multiple clinical concept identifiers with input data including embeddings that correspond to medication concepts, condition concepts, or other ontology-defined entities. In further examples, a prompt template may specify computation of derived values based on structured pharmacokinetic attribute data encoded within embeddings. In each case, the prompt templates define structured computational queries rather than free-form natural language interactions, and the ML model generates corresponding machine-readable relationship outputs.

In one or more embodiments, hierarchical parent-child relationships and other ontology-defined structures are incorporated into the ML model and/or embedding space. Such hierarchical structures may be encoded directly within the embeddings or provided to the ML model as auxiliary inputs that define permissible or weighted relationships among concepts. For example, broader-to-narrower concept relationships and cross-ontology mappings defined by resources, such as UMLS, may be used to inform classification, association, or linking operations performed by the model. Encoding hierarchical and ontology-based constraints in this manner enables the system to generate relationship data that is consistent with predefined structural representations while operating entirely within a computer-implemented processing framework.

One or more embodiments identify a new relationship (Operation 208). The system applies the trained ML model to vector embeddings that correspond to new data instances or combinations of data instances not previously paired during model training. In this manner, the model generates relationship data between items represented in the embedding space, such as associations between molecular entities, biomedical concepts, or other ontology-defined elements. The system may apply the model to expanded or updated datasets to extend previously derived relationships to additional data instances or to generalize learned patterns across larger data populations.

In one or more embodiments, the system analyzes outputs generated by the ML model to determine relative significance, confidence scores, or other quantitative measures associated with the predicted relationships. Such analysis may include evaluating model parameters, intermediate activation values, feature contribution scores, and/or other internal model metrics produced during inference. The system may apply post-processing or interpretability techniques to the model outputs to generate auxiliary data describing how particular vector embeddings or embedding features contributed to a given prediction. These auxiliary data may be generated using attribution or contribution analysis methods and may be stored as metadata associated with the predicted relationships.

In one or more embodiments, the system generates graphical or visual representations of the embedding space and the predicted relationships to support inspection, validation, or downstream computational use. For example, the system may apply dimensionality-reduction techniques to project high-dimensional vector embeddings into lower-dimensional spaces suitable for visualization. Such projections may be used to generate plots or graphs illustrating relative positions of embeddings and connectivity patterns among concepts or expressions. The visual representations are generated as computer-rendered outputs and may be stored, displayed, or transmitted as part of system operation without requiring manual interpretation or clinical judgment.

One or more embodiments generate vector embeddings for the new relationships (Operation 210). The system generates such relationship embeddings using one or more vector embedding functions applied to machine-readable representations of the relationship data produced by the trained model. In some embodiments, the relationship embeddings are generated using the same vector embedding functions previously applied to clinical expressions or genomic expressions to maintain representational consistency within a shared embedding space. In other embodiments, different vector embedding functions are selected to account for structural differences between entity embeddings and relationship embeddings.

In certain implementations, the system derives relationship embeddings from intermediate or final hidden layers of the ML model used to generate the relationship data. Such hidden-layer representations may encode learned interaction features between paired or grouped embeddings and may be extracted as fixed-length numerical vectors. The extracted vectors may be normalized, scaled, or otherwise transformed to satisfy dimensionality or compatibility constraints with other embeddings maintained by the system. The resulting relationship embeddings are stored as machine-readable data objects and may be indexed, cached, or provided as inputs to subsequent processing stages.

In one or more embodiments, the system generates composite vector representations for relationships by combining vector embeddings corresponding to related clinical concepts and genomic concepts. For example, the system may concatenate a clinical embedding and a genomic embedding identified as participating in a relationship by appending the respective vectors to form a unified numerical representation. In some embodiments, the system applies weighting factors to one or more portions of the concatenated vector to reflect relative relevance, confidence scores, or other model-generated metrics associated with the relationship.

In addition to direct concatenation, the system may generate composite embeddings using graph-based combination techniques that incorporate relational context derived from ontology or graph structures. For example, embeddings associated with neighboring nodes or edges in a concept graph may be aggregated, pooled, or otherwise combined to generate a relationship representation that captures both entity-level and structural information. The resulting composite embeddings are stored as numerical data structures suitable for computational comparison, indexing, or further ML-based processing.

One or more embodiments classify the vector embeddings using a classifier (Operation 212). The system applies the classifier to vector embeddings corresponding to expressions, concepts, or relationships to assign one or more class labels or category identifiers as machine-generated outputs. In some embodiments, the classifier is configured as a multi-output or multi-label classifier capable of producing multiple classification outputs for a single input embedding.

In embodiments implemented using neural network architectures, the classifier includes an input layer configured to accept vector embeddings having a dimensionality corresponding to the embedding space. One or more hidden layers may be included to transform the input embeddings, such as fully connected (dense) layers, normalization layers, or regularization layers including dropout layers. The classifier further includes multiple output layers, each corresponding to a respective classification dimension or label set. Each output layer may apply an activation function selected based on the classification configuration, such as a SoftMax activation for mutually exclusive classes or a sigmoid activation for independent or non-exclusive labels. The neural network classifier may be trained using backpropagation and gradient-based optimization techniques to minimize one or more loss functions associated with the classification outputs.

In other embodiments, the classifier is implemented using non-neural ML models provided by general-purpose ML libraries. For example, the system may employ wrapper-based classifier constructs configured to manage multiple output labels by coordinating multiple underlying base classifiers. Each base classifier may be trained to operate on the same vector embedding input while producing a distinct classification output. The wrapper construct manages parallel execution, aggregation of outputs, and consistency of input handling across the multiple classifiers.

The classification results generated by the classifier are produced as structured, machine-readable data objects, such as label vectors, probability distributions, or score arrays. These outputs may be stored, indexed, or forwarded to subsequent processing stages without requiring human interpretation. The classification operation is performed entirely within the computing system as a transformation of numerical input data into numerical or symbolic output representations.

In one or more embodiments, the classifier is trained using a labeled dataset in which each training instance is associated with a plurality of labels corresponding to multiple output dimensions. Each training instance includes one or more vector embeddings serving as input features, and the associated labels are provided as structured training targets. During training, the classifier executes a learning process in which parameters of the classifier are iteratively adjusted to map numerical features of the input embeddings to the corresponding label representations across the multiple output dimensions.

The training process may include computing loss values for each output dimension, aggregating or weighting such loss values, and applying optimization techniques to update model parameters. The classifier thereby learns statistical associations between patterns present in the embedding features and the label distributions encoded in the training dataset. The resulting trained classifier is configured to generate multi-dimensional classification outputs for previously unseen vector embeddings during subsequent inference operations with all training and inference operations performed as computer-implemented numerical transformations.

One or more embodiments train a multi-level classifier using the vector embeddings (Operation 214). The system may train the multi-level classifier using curated training datasets that include structured annotations linking textual or symbolic data to standardized concept identifiers. By way of example, curated data may include annotated clinical text segments that have been pre-associated with corresponding standardized clinical concept identifiers where such associations are represented as machine-readable labels. The system may further incorporate a) genomic datasets represented in structured formats and b) data derived from standardized knowledge bases and terminologies to provide labeled or semi-labeled training inputs for the classifier.

In some embodiments, the training datasets include concept identifiers and relationships derived from standardized resources, such as RxNorm, UMLS, ICD-10, and SNOMED CT. Data from these resources may be transformed into numerical or categorical label representations aligned with the hierarchical structure of the multi-level classifier. The classifier is trained to map input vector embeddings to corresponding label representations at multiple hierarchical levels with all training operations performed as computer-implemented numerical transformations.

In one or more embodiments, the system automatically trains the multi-level classifier using a stepwise or iterative refinement process. Such training may be implemented using deep learning architectures configured to learn hierarchical feature representations from the input vector embeddings. For example, intermediate layers of the classifier may be configured to encode progressively abstract or aggregated features that correspond to different hierarchy levels represented in the label space. Attributes, object properties, or relationship indicators encoded in the embeddings may be propagated through successive layers of the model to support multi-level classification.

In some embodiments, the classifier employs hierarchical output structures in which predictions at one level of the hierarchy constrain or inform predictions at lower or higher levels. For example, the system may apply hierarchical SoftMax or similar computational techniques to efficiently model large, structured label spaces while preserving hierarchical relationships among labels. The stepwise refinement process enables the classifier to be trained incrementally across hierarchy levels using gradient-based optimization with the resulting trained model configured to generate structured, multi-level classification outputs for input embeddings during inference.

One or more embodiments apply the multilevel classifier to a vector embedding representing a condition of a patient to identify hierarchies and relationships associated with the condition (Operation 216). The system may access patient-related data from an electronic health record (EHR) system or other clinical data repository where the accessed data include structured or unstructured text fields describing one or more recorded patient conditions or observations. The system generates one or more vector embeddings from the accessed text using a vector embedding function. The vector embedding function may be the same embedding function or a different embedding function than that the one used to generate embeddings for clinical concepts or genomic concepts. The generated vector embeddings are provided as inputs to the multi-level classifier, which produces structured outputs indicating positions within one or more predefined hierarchical classification structures and associated relationships encoded by the classifier.

In one or more embodiments, the multi-level classifier is applied to vector embeddings that correspond to data instances not previously encountered during training to generate classification outputs within the hierarchical label space. Such outputs may include assignments to hierarchy levels, confidence scores, similarity-based associations, or linkage indicators relative to existing labeled data instances. The generated outputs represent machine-generated classification data derived from learned statistical relationships within the embedding space and may be stored, indexed, or forwarded to downstream system components for further computational processing or analysis. The system does not require manual interpretation to generate these outputs.

In one or more embodiments, the system utilizes learned parameters of the trained multi-level classifier, such as weights associated with one or more layers of a neural network, to compute similarity or association metrics between vector embeddings. For example, weights from one or more final or intermediate layers of the classifier may be applied to transform input embeddings into comparison-ready representations. The system may compute similarity scores between transformed embeddings corresponding to new data instances and embeddings corresponding to existing concepts or relationships. These similarity scores are generated as numerical values that quantify relative proximity or association within the embedding space and may be used as machine-readable indicators for subsequent computational operations.

One or more embodiments treat the patient in accordance with the identified hierarchies and relationships associated with the condition (Operation 218). The system may provide outputs of the multi-level classifier to one or more downstream software applications, services, and/or user interfaces. These may include clinical information systems or decision-support applications. Such outputs may include structured classification data, relationship indicators, confidence scores, similarity metrics, and treatment-related recommendation data generated by the classifier.

In some embodiments, the system organizes, groups, ranks, and/or filters treatment options, intervention categories, diagnostic follow-up actions, and/or care pathways for presentation within a graphical user interface. For example, the system may generate ordered lists of medications, therapeutic classes, procedural options, or testing recommendations based on computed hierarchical classifications and relationship data. The presentation logic may be configured to prioritize or visually distinguish treatment recommendations that satisfy predefined clinical criteria, threshold values, or genomic association indicators computed by the system.

In one or more embodiments, the system generates alerts, notifications, and/or treatment recommendations when computed classification outputs or relationship metrics satisfy configurable conditions. Such alerts may correspond to identification of high-risk conditions, detection of genomic predisposition patterns, potential drug-response interactions, and/or progression indicators derived from statistical modeling. The alerts may be transmitted to clinician-facing systems, incorporated into workflow dashboards, or recorded within a patient record to prompt clinical review.

The system may further generate structured recommendation entries representing suggested treatments, medication adjustments, dosage modifications, diagnostic testing recommendations, preventive measures, and/or follow-up evaluations. In some embodiments, these recommendations are stored in an EHR system as structured data entries associated with the patient record. The system may automatically populate treatment suggestion fields or draft orders for clinician approval. The system may stage recommendations for review and confirmation by an authorized healthcare provider prior to execution.

In one or more embodiments, the system produces outputs reflecting predicted treatment response, projected disease or condition progression, comparative effectiveness of alternative therapeutic options, and/or risk-adjusted care recommendations derived from integrated clinical and genomic modeling. These outputs may be used to support personalized treatment planning and care management workflows.

### 4. Example System for Training a Multilevel Classifier

A detailed example is described below for purposes of clarity. Components and/or operations described below should be understood as one specific example that may not be applicable to certain embodiments. Accordingly, components and/or operations described below should not be construed as limiting the scope of any of the claims.

FIG. 3 illustrates an example embodiment of a system 300 for generating vector embeddings from heterogeneous data inputs and training a multilevel classifier 330 using hierarchical label outputs. System 300 includes a vector generator 302, a transformer-based model 304, a classifier 306, and a training module 308. As illustrated, system 300 is configured to process both clinical and genomic data, derive structured relationship representations, and use those representations to train multilevel classifier 330. The components of system 300 may be implemented as software modules executed by one or more hardware processors and may operate in conjunction with one or more data repositories storing training data and model parameters.

Initially, system 300 ingests a clinical expression 312 and genomic data 314. Clinical expression 312 may include structured clinical concept identifiers, pre-coordinated expressions, post-coordinated expressions, attributes, hierarchical descriptors, or unstructured clinical narrative text, such as “Elevated LDL cholesterol, Tendon xanthomas.” Genomic data 314 may include genomic feature indicators, such as “Gene Variant: rs12345,” nucleotide or amino acid sequences, structured variant annotations, zygosity indicators, or other genomic metadata. In one embodiment, clinical expression 312 and genomic data 314 are retrieved from an EHR system or a structured data repository and serialized into machine-readable training instance objects.

Clinical expression 312 and genomic data 314 are provided to vector generator 302. Vector generator 302 performs normalization and preprocessing operations on the input data. Such preprocessing may include converting text to lowercase, tokenizing text into words or sub words, retaining numeric tokens (e.g., laboratory measurement values), handling punctuation and special characters, removing unwanted tokens, expanding abbreviations, and applying domain-specific normalization rules. In embodiments in which the input includes structured identifiers or attributes, vector generator 302 may concatenate textual descriptions, attribute values, and hierarchical path descriptors into a unified representation prior to embedding.

Vector generator 302 then applies a first vector embedding function to the normalized representation of clinical expression 312 to generate clinical embedding 316. The first vector embedding function may be implemented using a contextual language model, a transformer-based encoder, a word embedding model, or another embedding architecture suitable for clinical text. Similarly, vector generator 302 applies a second vector embedding function to the normalized representation of genomic data 314 to generate genomic embedding 318. The second embedding function may be implemented using a sequence-aware embedding model, a k-mer-based encoder, or a feature transformation network. In some embodiments, the first and second embedding functions are identical; in other embodiments, they are distinct models selected based on the characteristics of the input data.

System 300 next applies transformer-based model 304 to clinical embedding 316 and genomic embedding 318 to identify new relationship 320. Transformer-based model 304 may implement self-attention mechanisms to capture interaction features between the clinical and genomic embeddings. In one embodiment, transformer-based model 304 concatenates clinical embedding 316 and genomic embedding 318 and applies one or more attention layers to compute contextualized interaction representations. New relationship 320 may include a similarity score, an interaction feature vector, a predicted relationship category, and/or a confidence value. The output of transformer-based model 304 is generated as a machine-readable data structure.

Vector generator 302 next generates relationship embedding 322 by applying a third vector embedding function to new relationship 320. In one embodiment, the third embedding function transforms interaction features or relationship descriptors into a fixed-length numerical vector suitable for classification. The third vector embedding function may be the same as (or different from) the first or second embedding functions. In some implementations, relationship embedding 322 is generated by projecting a concatenated vector of clinical embedding 316 and genomic embedding 318 into a lower-dimensional space. In other implementations, relationship embedding 322 is extracted from an intermediate hidden layer of transformer-based model 304.

System 300 then applies classifier 306 to each of clinical embedding 316, genomic embedding 318, and relationship embedding 322. Classifier 306 may include one or more fully connected layers, normalization layers, and output layers configured to generate hierarchical classification outputs. Classifier 306 identifies a first label 324 for clinical embedding 316, a second label 326 for relationship embedding 322, and a third label 328 for genomic embedding 318. First label 324, second label 326, and third label 328 correspond to hierarchical classification targets. For example, first label 324 may correspond to a first-level category (e.g., a broad disorder class), second label 326 may correspond to a second-level subcategory (e.g., a disorder subtype or interaction class), and third label 328 may correspond to a third-level specific concept identifier (e.g., a standardized code). The labels are structured targets associated with each training instance and may be derived from standardized hierarchies. The labels may be encoded as categorical identifiers, one-hot vectors, integer indices, or probability distributions.

Training module 308 receives clinical embedding 316, genomic embedding 318, and relationship embedding 322, together with corresponding first label 324, second label 326, and third label 328. Training module 308 coordinates the optimization of parameters for multilevel classifier 330. For each training instance, training module 308 computes predicted outputs for each hierarchical level and calculates a loss value by comparing predicted outputs to the corresponding structured targets. In one embodiment, a separate loss term is computed for each hierarchy level and aggregated into a composite loss function. Training module 308 updates classifier parameters using gradient-based optimization techniques to minimize the composite loss.

Training proceeds iteratively over multiple training instances or batches. During each iteration, embeddings are generated, interaction features are computed, hierarchical predictions are produced, loss values are calculated, and classifier parameters are adjusted. Regularization techniques, such as dropout, weight decay, or normalization, may be applied to improve generalization. Upon convergence or completion of training criteria, the optimized parameters are stored as part of trained multilevel classifier 330.

After training, trained multilevel classifier 330 is configured to receive embeddings that correspond to new data instances and generate structured hierarchical classification outputs. During inference, the classifier operates on numerical vector inputs, including clinical embeddings, genomic embeddings, and/or relationship embeddings, and produces machine-readable outputs corresponding to multiple hierarchical levels. The outputs may include hierarchical label identifiers and associated confidence values. Trained multilevel classifier 330 operates entirely within the computing environment, performing numerical transformations without requiring manual rule authoring or manual interpretation.

### 5. Machine Learning Architecture

FIG. 4 illustrates a machine learning engine 454 in accordance with one or more embodiments. As illustrated in FIG. 4, machine learning engine 454 includes input/output module 462, data preprocessing module 464, model selection module 466, training module 468, evaluation and tuning module 470, and inference module 472.

In accordance with an embodiment, input/output module 462 serves as the primary interface for data entering and exiting the system, managing the flow and integrity of data. This module may accommodate a wide range of data sources and formats to facilitate integration and communication within the machine learning architecture.

In an embodiment, an input handler within input/output module 462 includes a data ingestion framework capable of interfacing with various data sources, such as databases, APIs, file systems, and real-time data streams. This framework is equipped with functionalities to handle different data formats (e.g., CSV, JSON, XML) and efficiently manage large volumes of data. This includes mechanisms for batch and real-time data processing that enable the input/output module 462 to be versatile in different operational contexts, whether processing historical datasets or streaming data.

In accordance with an embodiment, input/output module 462 manages data integrity and quality as the data enters the system by incorporating initial checks and validations. These checks and validations ensure that incoming data meets predefined quality standards, like checking for missing values, ensuring consistency in data formats, and verifying data ranges and types. This proactive approach to data quality minimizes potential errors and inconsistencies in later stages of the machine learning process.

In an embodiment, an output handler within input/output module 462 includes an output framework designed to handle the distribution and exportation of outputs, predictions, or insights. Using the output framework, input/output module 462 formats these outputs into user-friendly and accessible formats, such as reports, visualizations, or data files compatible with other systems. Input/output module 462 also ensures secure and efficient transmission of these outputs to end-users or other systems in an embodiment and may employ encryption and secure data transfer protocols to maintain data confidentiality.

In accordance with an embodiment, data preprocessing module 464 transforms data into a format suitable for use by other modules in machine learning engine 454. For example, data preprocessing module 464 may transform raw data into a normalized or standardized format suitable for training ML models and for processing new data inputs for inference. In an embodiment, data preprocessing module 464 acts as a bridge between the raw data sources and the analytical capabilities of machine learning engine 454.

In an embodiment, data preprocessing module 464 begins by implementing a series of preprocessing steps to clean, normalize, and/or standardize the data. This involves handling a variety of anomalies, such as managing unexpected data elements, recognizing inconsistencies, or dealing with missing values. Some of these anomalies can be addressed through methods like imputation or removal of incomplete records, depending on the nature and volume of the missing data. Data preprocessing module 464 may be configured to handle anomalies in different ways depending on context. Data preprocessing module 464 also handles the normalization of numerical data in preparation for use with models sensitive to the scale of the data, like neural networks and distance-based algorithms. Normalization techniques, such as min-max scaling or z-score standardization, may be applied to bring numerical features to a common scale, enhancing the model's ability to learn effectively.

In an embodiment, data preprocessing module 464 includes a feature encoding framework that ensures categorical variables are transformed into a format that can be easily interpreted by machine learning algorithms. Techniques like one-hot encoding or label encoding may be employed to convert categorical data into numerical values, making them suitable for analysis. The module may also include feature selection mechanisms, where redundant or irrelevant features are identified and removed, thereby increasing the efficiency and performance of the model.

In accordance with an embodiment, when data preprocessing module 464 processes new data for inference, data preprocessing module 464 replicates the same preprocessing steps to ensure consistency with the training data format. This helps to avoid discrepancies between the training data format and the inference data format, thereby reducing the likelihood of inaccurate or invalid model predictions.

In an embodiment, model selection module 466 includes logic for determining the most suitable algorithm or model architecture for a given dataset and problem. This module operates in part by analyzing the characteristics of the input data, such as its dimensionality, distribution, and the type of problem (classification, regression, clustering, etc.).

In an embodiment, model selection module 466 employs a variety of statistical and analytical techniques to understand data patterns, identify potential correlations, and assess the complexity of the task. Based on this analysis, model selection module 466 then matches the data characteristics with the strengths and weaknesses of various available models. This can range from simple linear models for less complex problems to sophisticated deep learning architectures for tasks requiring feature extraction and high-level pattern recognition, such as image and speech recognition.

In an embodiment, model selection module 466 utilizes techniques from the field of Automated Machine Learning (AutoML). AutoML systems automate the process of model selection by rapidly prototyping and evaluating multiple models. They use techniques like Bayesian optimization, genetic algorithms, or reinforcement learning to explore the model space efficiently. Model selection module 466 may use these techniques to evaluate each candidate model based on performance metrics relevant to the task. For example, accuracy, precision, recall, or F1 score may be used for classification tasks and mean squared error metrics may be used for regression tasks. Accuracy measures the proportion of correct predictions (both positive and negative). Precision measures the proportion of actual positives among the predicted positive cases. Recall (also known as sensitivity) evaluates how well the model identifies actual positives. F1 Score is a single metric that accounts for both false positives and false negatives. The mean squared error (MSE) metric may be used for regression tasks. MSE measures the average squared difference between the actual and predicted values, providing an indication of the model's accuracy. A lower MSE may indicate a model's greater accuracy in predicting values, as it represents a smaller average discrepancy between the actual and predicted values.

In accordance with an embodiment, model selection module 466 also considers computational efficiency and resource constraints. This is meant to help ensure the selected model is both accurate and practical in terms of computational and time requirements. In an embodiment, certain features of model selection module 466 are configurable such as a configured bias toward (or against) computational efficiency.

In accordance with an embodiment, training module 468 manages the ‘learning’ process of ML models by implementing various learning algorithms that enable models to identify patterns and make predictions or decisions based on input data. In an embodiment, the training process begins with the preparation of the dataset after preprocessing; this involves splitting the data into training and validation sets. The training set is used to teach the model, while the validation set is used to evaluate its performance and adjust parameters accordingly. Training module 468 handles the iterative process of feeding the training data into the model, adjusting the model's internal parameters (like weights in neural networks) through backpropagation and optimization algorithms, such as stochastic gradient descent or other algorithms providing similarly useful results.

In accordance with an embodiment, training module 468 manages overfitting, where a model learns the training data too well, including its noise and outliers, at the expense of its ability to generalize to new data. Techniques such as regularization, dropout (in neural networks), and early stopping are implemented to mitigate this. Additionally, the module employs various techniques for hyperparameter tuning; this involves adjusting model parameters that are not directly learned from the training process, such as learning rate, the number of layers in a neural network, or the number of trees in a random forest.

In an embodiment, training module 468 includes logic to handle different types of data and learning tasks. For instance, it includes different training routines for supervised learning (where the training data comes with labels) and unsupervised learning (without labeled data). In the case of deep learning models, training module 468 also manages the complexities of training neural networks that include initializing network weights, choosing activation functions, and setting up neural network layers.

In an embodiment, evaluation and tuning module 470 incorporates dynamic feedback mechanisms and facilitates continuous model evolution to help ensure the system's relevance and accuracy as the data landscape changes. Evaluation and tuning module 470 conducts a detailed evaluation of a model's performance. This process involves using statistical methods and a variety of performance metrics to analyze the model's predictions against a validation dataset. The validation dataset, distinct from the training set, is instrumental in assessing the model's predictive accuracy and its capacity to generalize beyond the training data. The module's algorithms meticulously dissect the model's output, uncovering biases, variances, and the overall effectiveness of the model in capturing the underlying patterns of the data.

In an embodiment, evaluation and tuning module 470 performs continuous model tuning by using hyperparameter optimization. Evaluation and tuning module 470 performs an exploration of the hyperparameter space using algorithms, such as grid search, random search, or more sophisticated methods like Bayesian optimization. Evaluation and tuning module 470 uses these algorithms to iteratively adjust and refine the model's hyperparameters-settings that govern the model's learning process but are not directly learned from the data-to enhance the model's performance. This tuning process helps to balance the model's complexity with its ability to generalize and attempts to avoid the pitfalls of underfitting or overfitting.

In an embodiment, evaluation and tuning module 470 integrates data feedback and updates the model. Evaluation and tuning module 470 actively collects feedback from the model's real-world applications, an indicator of the model's performance in practical scenarios. Such feedback can come from various sources depending on the nature of the application. For example, in a user-centric application like a recommendation system, feedback might comprise user interactions, preferences, and responses. In other contexts, such as predicting events, it might involve analyzing the model's prediction errors, misclassifications, or other performance metrics in live environments.

In an embodiment, feedback integration logic within evaluation and tuning module 470 integrates this feedback using a process of assimilating new data patterns, user interactions, and error trends into the system's knowledge base. The feedback integration logic uses this information to identify shifts in data trends or emergent patterns that were not present or inadequately represented in the original training dataset. Based on this analysis, the module triggers a retraining or updating cycle for the model. If the feedback suggests minor deviations or incremental changes in data patterns, the feedback integration logic may employ incremental learning strategies, fine-tuning the model with the new data while retaining its previously learned knowledge. In cases where the feedback indicates significant shifts or the emergence of new patterns, a more comprehensive model updating process may be initiated. This process might involve revisiting the model selection process, re-evaluating the suitability of the current model architecture, and/or potentially exploring alternative models or configurations that are more attuned to the new data.

In accordance with an embodiment, throughout this iterative process of feedback integration and model updating, evaluation and tuning module 470 employs version control mechanisms to track changes, modifications, and the evolution of the model, facilitating transparency and allowing for rollback if necessary. This continuous learning and adaptation cycle, driven by real-world data and feedback, helps to endure the model's ongoing effectiveness, relevance, and accuracy.

In an embodiment, inference module 472 transforms data raw data into actionable, precise, and contextually relevant predictions. In addition to processing and applying a trained model to new data, inference module 472 may also include post-processing logic that refines the raw outputs of the model into meaningful insights.

In an embodiment, inference module 472 includes classification logic that takes the probabilistic outputs of the model and converts them into definitive class labels. This process involves an analytical interpretation of the probability distribution for each class. For example, in binary classification, the classification logic may identify the class with a probability above a certain threshold, but classification logic may also consider the relative probability distribution between classes to create a more nuanced and accurate classification.

In an embodiment, inference module 472 transforms the outputs of a trained model into definitive classifications. Inference module 472 employs the underlying model as a tool to generate probabilistic outputs for each potential class. It then engages in an interpretative process to convert these probabilities into concrete class labels.

In an embodiment, when inference module 472 receives the probabilistic outputs from the model, it analyzes these probabilities to determine how they are distributed across some or every potential class. If the highest probability is not significantly greater than the others, inference module 472 may determine that there is ambiguity or interpret this as a lack of confidence displayed by the model.

In an embodiment, inference module 472 uses thresholding techniques for applications where making a definitive decision based on the highest probability might not suffice due to the critical nature of the decision. In such cases, inference module 472 assesses if the highest probability surpasses a certain confidence threshold that is predetermined based on the specific requirements of the application. If the probabilities do not meet this threshold, inference module 472 may flag the result as uncertain or defer the decision to a human expert. Inference module 472 dynamically adjusts the decision thresholds based on the sensitivity and specificity requirements of the application, subject to calibration for balancing the trade-offs between false positives and false negatives.

In accordance with an embodiment, inference module 472 contextualizes the probability distribution against the backdrop of the specific application. This involves a comparative analysis, especially in instances where multiple classes have similar probability scores, to deduce the most plausible classification. In an embodiment, inference module 472 may incorporate additional decision-making rules or contextual information to guide this analysis, ensuring that the classification aligns with the practical and contextual nuances of the application.

In regression models, where the outputs are continuous values, inference module 472 may engage in a detailed scaling process in an embodiment. Outputs, often normalized or standardized during training for optimal model performance, are rescaled back to their original range. This rescaling involves recalibration of the output values using the original data's statistical parameters, such as mean and standard deviation, ensuring that the predictions are meaningful and comparable to the real-world scales they represent.

In an embodiment, inference module 472 incorporates domain-specific adjustments into its post-processing routine. This involves tailoring the model's output to align with specific industry knowledge or contextual information. For example, in financial forecasting, inference module 472 may adjust predictions based on current market trends, economic indicators, or recent significant events, ensuring that the outputs are both statistically accurate and practically relevant.

In an embodiment, inference module 472 includes logic to handle uncertainty and ambiguity in the model's predictions. In cases where inference module 472 outputs a measure of uncertainty, such as in Bayesian inference models, inference module 472 interprets these uncertainty measures by converting probabilistic distributions or confidence intervals into a format that can be easily understood and acted upon. This provides users with both a prediction and an insight into the confidence level of that prediction. In an embodiment, inference module 472 includes mechanisms for involving human oversight or integrating the instance into a feedback loop for subsequent analysis and model refinement.

In an embodiment, inference module 472 formats the final predictions for end-user consumption. Predictions are converted into visualizations, user-friendly reports, or interactive interfaces. In some systems, like recommendation engines, inference module 472 also integrates feedback mechanisms, where user responses to the predictions are used to continually refine and improve the model, creating a dynamic, self-improving system.

FIG. 4 illustrates the operation of a machine learning engine in one or more embodiments. In an embodiment, input/output module 462 receives a dataset intended for training (Operation 401). This data can originate from diverse sources, like databases or real-time data streams, and in varied formats, such as CSV, JSON, or XML. Input/output module 462 assesses and validates the data, ensuring its integrity by checking for consistency, data ranges, and types.

In an embodiment, training data is passed to data preprocessing module 464. Here, the data undergoes a series of transformations to standardize and clean it, making it suitable for training ML models (Operation 402). This involves normalizing numerical data, encoding categorical variables, and handling missing values through techniques like imputation.

In an embodiment, prepared data from the data preprocessing module 464 is then fed into model selection module 466 (Operation 403). This module analyzes the characteristics of the processed data, such as dimensionality and distribution, and selects the most appropriate model architecture for the given dataset and problem. It employs statistical and analytical techniques to match the data with an optimal model, ranging from simpler models for less complex tasks to more advanced architectures for intricate tasks.

In an embodiment, training module 468 trains the selected model with the prepared dataset (Operation 404). It implements learning algorithms to adjust the model's internal parameters, optimizing them to identify patterns and relationships in the training data. Training module 468 also addresses the challenge of overfitting by implementing techniques, like regularization and early stopping, ensuring the model's generalizability.

In an embodiment, evaluation and tuning module 470 evaluates the trained model's performance using the validation dataset (Operation 405). Evaluation and tuning module 470 applies various metrics to assess predictive accuracy and generalization capabilities. It then tunes the model by adjusting hyperparameters, and if needed, incorporates feedback from the model's initial deployments, retraining the model with new data patterns identified from the feedback.

In an embodiment, input/output module 462 receives a dataset intended for inference. Input/output module 462 assesses and validates the data (Operation 406).

In an embodiment, data preprocessing module 464 receives the validated dataset intended for inference (Operation 407). Data preprocessing module 464 ensures that the data format used in training is replicated for the new inference data, maintaining consistency and accuracy for the model's predictions.

In an embodiment, inference module 472 processes the new data set intended for inference, using the trained and tuned model (Operation 408). It applies the model to this data, generating raw probabilistic outputs for predictions. Inference module 472 then executes a series of post-processing steps on these outputs, such as converting probabilities to class labels in classification tasks or rescaling values in regression tasks. It contextualizes the outputs as per the application's requirements, handling any uncertainty in predictions and formatting the final outputs for end-user consumption or integration into larger systems.

In an embodiment, machine learning engine API 474 allows for applications to leverage machine learning engine 454. In an embodiment, machine learning engine API 474 may be built on a RESTful architecture and offer stateless interactions over standard HTTP/HTTPS protocols. Machine learning engine API 474 may feature a variety of endpoints, each tailored to a specific function within machine learning engine 454. In an embodiment, endpoints such as /submitData facilitate the submission of new data for processing, while /retrieveResults is designed for fetching the outcomes of data analysis or model predictions. The MLE API may also include endpoints like /updateModel for model modifications and /trainModel to initiate training with new datasets.

In an embodiment, machine learning engine API 474 is equipped to support SOAP-based interactions. This extension involves defining a WSDL (Web Services Description Language) document that outlines the API's operations and the structure of request and response messages. In an embodiment, machine learning engine API 474 supports various data formats and communication styles. In an embodiment, machine learning engine API 474 endpoints may handle requests in JSON format or any other suitable format. For example, machine learning engine API 474 may process XML, and it may also be engineered to handle more compact and efficient data formats, such as Protocol Buffers or Avro, for use in bandwidth-limited scenarios.

In an embodiment, machine learning engine API 474 is designed to integrate WebSocket technology for applications necessitating real-time data processing and immediate feedback. This integration enables a continuous, bi-directional communication channel for a dynamic and interactive data exchange between the application and machine learning engine 454.

### 6. Generative Models

A generative model is a machine learning model that is capable of generating new data instances based on the data used to train the model. A generative model may be referred to as a “generative artificial intelligence (AI) model.” Generative models learn the underlying distribution of the training data, enabling them to produce new instances of data that share properties with the original dataset. This capability makes them particularly useful in a variety of applications, including image and voice generation, text synthesis, and more sophisticated tasks like unsupervised learning, semi-supervised learning, and domain adaptation.

One type of generative model is a large language model. Large language models are designed to understand, generate, and interpret human language by processing extensive collections of data. The foundational architecture behind large language models is the transformer network, a type of neural network that excels in handling sequential data such as text. Unlike architectures, such as recurrent neural networks (RNNs) or long short-term memory networks (LSTMs), transformers do not process data in order. Instead, they leverage parallel processing to analyze entire text sequences simultaneously, significantly improving efficiency and reducing training times.

In an embodiment, a mechanism that enables transformers to handle complex language tasks is self-attention. This mechanism allows the model to weigh the importance of different words within a sentence or sequence regardless of their position. For instance, in processing the phrase “The cat sat on the mat,” the model can directly associate “cat” with “mat” without having to process the intermediate words sequentially. This ability to understand the context and relationships between words in a sentence is what makes transformer networks adept at language tasks. The self-attention mechanism assigns scores to relationships between words, highlighting the most relevant connections, so the model can focus on the most informative parts of the text.

In accordance with one or more embodiments, transformers are composed of multiple layers containing a multi-head, self-attention mechanism and a position-wise, feed-forward network. Within the architecture of transformer models, the multi-head, self-attention mechanism and position-wise, feed-forward network function in concert to process input data. The multi-head, self-attention mechanism is designed to enable parallel processing of input sequences, allowing the model to simultaneously evaluate the importance of different segments of the input relative to each other. This mechanism operates by generating multiple sets of query, key, and value vectors for each element in the input sequence through linear transformation. The relevance of each element to every other element is calculated using a scaled dot-product attention function that computes the attention scores by taking the dot product of the query vector with the key vectors, dividing each by the square root of the dimension of the key vectors to scale the scores, then applying a SoftMax function to obtain the weights for the value vectors. The scaled dot-product attention function is applied independently by each head in the multi-head self-attention mechanism. The outputs of these heads are then concatenated and linearly transformed, allowing the model to capture information from different representation subspaces.

In accordance with one or more embodiments, following the multi-head, self-attention mechanism is the position-wise, feed-forward network. This component comprises two linear transformations with a non-linear activation function in between. Each element of the input sequence, now enriched with context by the self-attention mechanism, is processed independently through the same feed-forward network. The first linear transformation increases the dimensionality of the input, allowing for a richer representation space. The non-linear activation function introduces the capability to capture non-linear relationships within the data. The second linear transformation then reduces the dimensionality back to that of the model's hidden layers, preparing the output for either further processing by subsequent layers or final output generation. This sequence of operations is applied to each position in the sequence, so the model can learn complex patterns across different parts of the input data without relying on the sequential processing inherent to previous architectures, such as RNNs or LSTMs.

In accordance with one or more embodiments, integrating these components within the transformer architecture facilitates the model's ability to understand and generate human language by leveraging both the global context provided by the self-attention mechanism and the local, position-specific transformations applied by the feed-forward networks. Through the repetitive stacking of layers, transformers achieve a depth of representation that allows for the processing of linguistic information across varying levels of complexity.

In accordance with one or more embodiments, input/output module 462, when used for large language models, handles textual data, converting input text into a format that the model can process. This typically involves tokenization, where the text is broken down into manageable pieces, such as words or sub words, and then converted into numerical representations. These representations, or embeddings, capture semantic information about the text that is then fed into the model for processing. The output from the model is converted from numerical form back into human-readable text, following the generation of predictions or responses.

In accordance with one or more embodiments, data preprocessing module 464 in the context of large language models may include steps such as normalization, where the text is converted to a uniform case and punctuation is standardized. This process ensures that the model treats similar words or symbols consistently, reducing the complexity of the input space. Additionally, techniques such as sentence segmentation may be applied to manage longer texts, enabling the model to process information in chunks that align with natural language structures.

In accordance with one or more embodiments, model selection module 466, when used for large language models involves choosing a specific architecture and configuration that is best suited to the task at hand. This decision is based on various factors, such as the size of the available training data, the complexity of the language tasks to be performed, and computational resource constraints. Models may vary in size from millions to billions of parameters, with larger models generally capable of more nuanced language understanding and generation but requiring significantly more computational power to train and operate.

In accordance with one or more embodiments, training module 468, when used for large language models, is configured to adjust the model's parameters through exposure to training data. This process utilizes optimization algorithms, such as stochastic gradient descent, to minimize the difference between the model's predictions and the actual desired outputs. The training process is computationally intensive, often requiring specialized hardware such as GPUs (Graphics Processing Units) or TPUs (Tensor Processing Units) to manage the large volumes of data and the complexity of the model calculations. During training, techniques, such as dropout and layer normalization, are used to improve model generalization and prevent overfitting (i.e., when a model learns the detail and noise in the training data to the extent that it negatively impacts the model's performance on new data).

In accordance with one or more embodiments, evaluation and tuning module 470 assesses the performance of large language models using metrics such as perplexity, accuracy, and F1 score, depending on the specific language tasks. Evaluation may involve comparing the model's output against a set of labeled validation data, providing insight into how well the model has learned to perform tasks, such as text classification, question answering, or text generation. Tuning involves adjusting model parameters or training strategies based on evaluation outcomes to improve performance. This may include hyperparameter tuning, where parameters that govern the training process, such as learning rate or batch size, are adjusted.

In accordance with one or more embodiments, inference module 472, in the context of large language models, is responsible for generating predictions or responses based on new, unseen data. This process involves feeding the input data through the trained model to produce an output. Inference can be used for a variety of applications, including translating text, generating human-like responses in a chatbot, or summarizing articles.

Another type of generative model is a large multimodal model (LMM). A large multimodal model is an advanced machine learning model capable of processing and generating data across multiple modalities, such as text, images, audio, and video. These models integrate diverse datasets during training to learn the underlying distribution of different data types, enabling them to produce outputs that reflect a comprehensive understanding of the input data. These models can be used for applications such as image captioning, text-to-image generation, image-to-text generation, visual question answering, and more, where understanding the relationship between different data types is crucial. By leveraging diverse datasets during training, large multimodal models learn to create coherent and contextually relevant outputs across various modalities, enhancing their utility in complex, real-world scenarios.

The architecture of large multimodal models combines elements from different neural network designs to handle diverse data types effectively. For example, convolutional neural networks (CNNs) are often used for processing visual data, while transformer networks handle textual data, enabling the model to extract and synthesize features from both images and text. This integration results in outputs that accurately represent the input data, reflecting a deep understanding of both modalities. The transformer architecture, known for its ability to manage sequential data, is frequently adapted to work alongside CNNs, allowing these models to benefit from the strengths of each neural network type.

The self-attention mechanism, which is part of a transformer network, enables the model to weigh the importance of different elements within an input sequence, regardless of their position. This allows the model to capture intricate relationships between various data types. For example, in an image captioning task, the model can associate specific visual features with corresponding descriptive text, enhancing the coherence and accuracy of the generated captions. By assigning scores to relationships between elements, the self-attention mechanism highlights the most relevant connections, enabling the model to focus on the most informative parts of the input data and perform complex multimodal tasks effectively.

In large multimodal models, data preprocessing is a step that ensures the input data is in a suitable format for the model to process. This involves tasks such as tokenization for text data, where the text is broken down into manageable pieces, and feature extraction for image data, where key visual elements are identified and encoded. By standardizing and normalizing different data types, preprocessing reduces the complexity of the input space, enabling the model to treat similar elements consistently. Effective preprocessing is essential for the model to integrate information from various modalities and produce accurate, meaningful outputs.

Training large multimodal models involves optimizing their parameters through exposure to diverse datasets that include paired data from different modalities. This computationally intensive process often requires specialized hardware like GPUs or TPUs to manage the large volumes of data and the complexity of the model calculations. Techniques such as dropout and layer normalization are employed to improve model generalization and prevent overfitting. By iteratively adjusting the model's parameters, the training process enables the model to learn underlying patterns and relationships within the data, enhancing its ability to generate coherent and contextually relevant outputs across different modalities.

Evaluation and tuning of large multimodal models are conducted using various metrics tailored to the specific tasks they are designed to perform. For example, BLEU scores are used for text generation tasks, while accuracy is commonly applied for visual recognition tasks to assess performance. Tuning involves adjusting hyperparameters and refining training strategies based on evaluation results to enhance the model's effectiveness. This iterative process ensures that the model can perform a wide range of multimodal tasks with high accuracy and relevance, making it a versatile tool for applications requiring the integration of different types of data.

Large multimodal models represent a significant advancement in machine learning by leveraging sophisticated architectures that combine different neural network types and apply self-attention mechanisms. This enables them to perform complex tasks that require understanding and synthesizing information from diverse data types. Effective preprocessing, rigorous training, and thorough evaluation are crucial to their success, allowing these models to generate coherent and contextually relevant outputs across a wide range of applications.

In accordance with one or more embodiments, other types of models besides large language models and large multimodal models belong to the broad category of generative models. For example, stochastic models directly incorporate randomness into their structure, making them inherently generative as they can produce a diverse set of outputs for a given input. Generative Adversarial Networks (GANs) learn to generate new data that is indistinguishable from the data they were trained on, using a dual-network architecture that involves a generative component. Variational Autoencoders (VAEs) are explicitly designed for generating new data points by learning a distribution of the input data and encode inputs into a latent space and generate outputs by sampling from this space, making them inherently generative. Sequence-to-sequence models are generative in nature when used with sampling strategies. Although this list of generative model types is not exhaustive, it illustrates the broad use of the term generative model beyond large language models.

Although generative models can be leveraged for classification tasks, they inherently operate on principles of randomness, leading to a spectrum of possible outcomes in response to identical inputs. Unlike deterministic models that yield a consistent result whenever the same input is given, generative models use the randomness in the data they are trained on to both mimic and diversify from the training data. This diversity makes generative models ideal for generating new and varied data points as well as for tasks that require creativity and novelty. However, a reliance on randomness creates a trade-off between predictability and flexibility for generative models, potentially making them less predictable in scenarios where uniform outcomes may be expected such as classification tasks.

### 7. Practical Applications, Advantages, and Improvements

Embodiments provide several practical applications, advantages, and improvements over existing computer-implemented approaches for processing, representing, and analyzing heterogeneous healthcare-related data. These advantages and improvements include enhanced computational representation of complex concept relationships, improved scalability and efficiency of machine-learning-based classification systems, and increased consistency and reliability of automated data processing across clinical and genomic data sources.

One or more embodiments enable computer systems to generate, transform, and operate on structured numerical representations of clinical and genomic information using vector embeddings, hierarchical classifiers, and ontology-informed models. These capabilities allow computing systems to perform large-scale, automated analysis of complex datasets in a manner that improves system performance, extensibility, and interoperability across heterogeneous data environments.

One or more embodiments improve the accuracy with which computer systems represent clinical and genomic concepts by generating vector embeddings that encode attributes, hierarchical relationships, and cross-domain associations. For example, by embedding both pre-coordinated and post-coordinated expressions together with ontology-derived relationships into a shared embedding space, the system produces representations that preserve semantic structure in a form suitable for numerical computation, thereby improving downstream classification and similarity operations.

One or more embodiments increase efficiency by enabling automated, ML-based processing of high-dimensional clinical and genomic datasets using multi-level classifiers and optimized embedding techniques. This results in reduced processing time and improved scalability when operating over large ontologies or expanding datasets. For instance, hierarchical SoftMax and multi-output classifier architectures allow the system to efficiently manage large label spaces without requiring exhaustive enumeration of all possible classifications.

One or more embodiments enhance interoperability by transforming heterogeneous clinical texts, genomic sequences, and ontology structures into standardized vector representations and structured classification outputs. For instance, data originating from different terminology systems or genomic datasets can be processed using common embedding and classification pipelines, reducing inconsistencies caused by format differences and enabling uniform downstream processing.

One or more embodiments improve upon existing approaches by reducing manual intervention and ad hoc data transformation steps through automated generation, classification, and management of vector embeddings and relationship data.

One or more embodiments provide a technical benefit by enabling computer systems to operate directly on numerical representations of complex healthcare data, resulting in improved processing performance, reduced system complexity, and more efficient use of computing resources. This is accomplished by replacing rule-based or hard-coded data handling logic with learned, data-driven models that generalize across datasets.

One or more embodiments implementing automated hierarchical classification of embedding-based data enable efficient organization and retrieval of structured information across multiple abstraction levels. This improves system responsiveness and supports scalable analytics by allowing downstream components to query and operate on precomputed classification outputs rather than raw data.

One or more embodiments improve the functioning of a computer system by enabling automated generation, classification, and dissemination of structured relationship data derived from heterogeneous clinical and genomic inputs. This improvement provides a technical benefit by reducing the computational overhead associated with repeated data parsing and transformation while improving the reliability of downstream data processing pipelines. This practical application improves upon existing approaches by integrating embedding generation, hierarchical modeling, and classification into a unified, computer-implemented framework.

In one or more embodiments, the system generates composite embeddings that encode relationships between clinical and genomic concepts and classifies such embeddings using a multi-level classifier. This improvement enables efficient reuse of learned representations across multiple processing stages and supports incremental system updates without retraining from scratch. For example, newly ingested data instances can be embedded and classified using existing models, reducing retraining costs and improving system uptime.

By providing structured, machine-readable classification outputs and relationship representations, embodiments enable downstream systems to perform analytics, visualization, indexing, and event generation operations more efficiently. This results in improved overall system performance and facilitates integration with other computing systems, user interfaces, and data repositories without requiring custom adaptation for each data source.

The data input to any ML model and/or the data output from any ML model, as described herein, may be used for operations performed by one or more of the following types of software: database, cloud infrastructure, customer relationship management, data science, digital assistant, vision, language, speech, forecasting, enterprise, middleware, server, identity management, application development, analytics, security, data integration, health, hospitality, retail, utilities, operating systems, virtualization, governance and administration, migration & disaster recovery, networking, connectivity, monitoring, procurement, project management, risk management, supply chain management, manufacturing, human capital management, customer experience, advertising, and industry-specific application.

### 8. Hardware Overview

According to one embodiment, the techniques described herein are implemented by one or more special-purpose computing devices. The special-purpose computing devices may be hard-wired to perform the techniques, or may include digital electronic devices such as one or more application-specific integrated circuits (ASICs), field programmable gate arrays (FPGAs), or network processing units (NPUs) that are persistently programmed to perform the techniques, or may include one or more general purpose hardware processors programmed to perform the techniques pursuant to program instructions in firmware, memory, other storage, or a combination. Such special-purpose computing devices may also combine custom hard-wired logic, ASICs, FPGAs, or NPUs with custom programming to accomplish the techniques. The special-purpose computing devices may be desktop computer systems, portable computer systems, handheld devices, networking devices or any other device that incorporates hard-wired and/or program logic to implement the techniques.

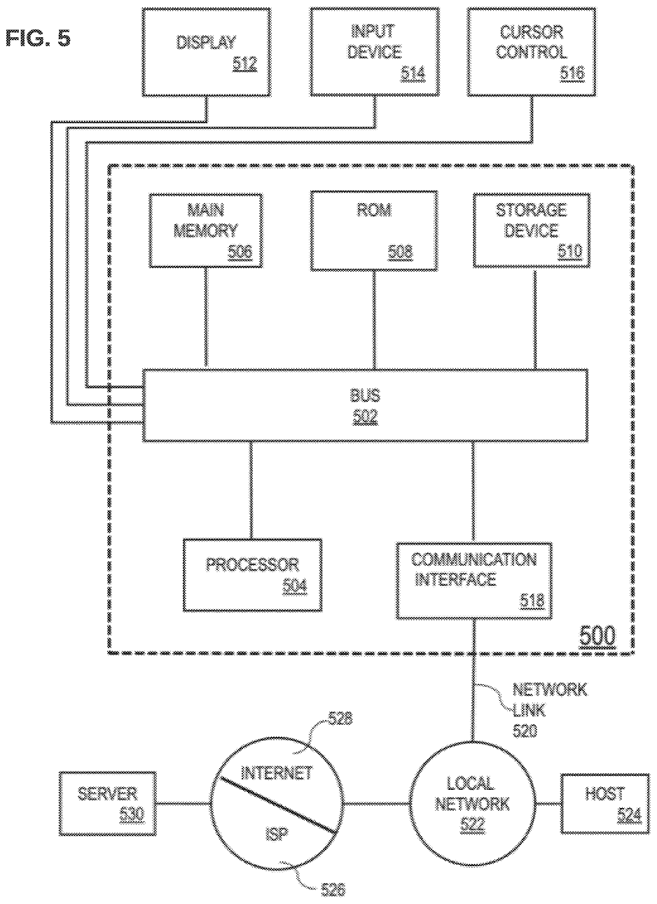

For example, FIG. 5 is a block diagram that illustrates a computer system 500 upon which an embodiment of the disclosure may be implemented. Computer system 500 includes a bus 502 or other communication mechanism for communicating information, and a hardware processor 504 coupled with bus 502 for processing information. Hardware processor 504 may be, for example, a general-purpose microprocessor.

Computer system 500 also includes a main memory 506, such as a random access memory (RAM) or other dynamic storage device, coupled to bus 502 for storing information and instructions to be executed by processor 504. Main memory 506 also may be used for storing temporary variables or other intermediate information during execution of instructions to be executed by processor 504. Such instructions, when stored in non-transitory storage media accessible to processor 504, render computer system 500 into a special-purpose machine that is customized to perform the operations specified in the instructions.

Computer system 500 further includes a read-only memory (ROM) 508 or other static storage device coupled to bus 502 for storing static information and instructions for processor 504. A storage device 510, such as a magnetic disk, optical disk, or a solid-state Drive (SSD) is provided and coupled to bus 502 for storing information and instructions.

Computer system 500 may be coupled via bus 502 to a display 512, such as a cathode ray tube (CRT), for displaying information to a computer user. An input device 514, including alphanumeric and other keys, is coupled to bus 502 for communicating information and command selections to processor 504. Another type of user input device is cursor control 516, such as a mouse, a trackball, or cursor direction keys for communicating direction information and command selections to processor 504 and for controlling cursor movement on display 512. This input device typically has two degrees of freedom in two axes, a first axis (e.g., x) and a second axis (e.g., y), that allows the device to specify positions in a plane.

Computer system 500 may implement the techniques described herein using customized hard-wired logic, one or more ASICs or FPGAs, firmware and/or program logic which in combination with the computer system causes or programs computer system 500 to be a special-purpose machine. According to one embodiment, the techniques herein are performed by computer system 500 in response to processor 504 executing one or more sequences of one or more instructions contained in main memory 506. Such instructions may be read into main memory 506 from another storage medium, such as storage device 510. Execution of the sequences of instructions contained in main memory 506 causes processor 504 to perform the process steps described herein. In alternative embodiments, hard-wired circuitry may be used in place of or in combination with software instructions.

The term “storage media” as used herein refers to any non-transitory media that store data and/or instructions that cause a machine to operate in a specific fashion. Such storage media may comprise non-volatile media and/or volatile media. Non-volatile media includes, for example, optical or magnetic disks, such as storage device 510. Volatile media includes dynamic memory, such as main memory 506. Common forms of storage media include, for example, a floppy disk, a flexible disk, hard disk, SSD, magnetic tape, or any other magnetic data storage medium, a CD-ROM, any other optical data storage medium, any physical medium with patterns of holes, a RAM, a PROM, and EPROM, a FLASH-EPROM, NVRAM, any other memory chip or cartridge, content-addressable memory (CAM), and ternary content-addressable memory (TCAM).

Storage media is distinct from but may be used in conjunction with transmission media. Transmission media participates in transferring information between storage media. For example, transmission media includes coaxial cables, copper wire and fiber optics, including the wires that comprise bus 502. Transmission media can also take the form of acoustic or light waves, such as those generated during radio-wave and infrared data communications.

Various forms of media may be involved in carrying one or more sequences of one or more instructions to processor 504 for execution. For example, the instructions may initially be carried on a magnetic disk or SSD of a remote computer. The remote computer can load the instructions into its dynamic memory and send the instructions over a telephone line using a modem. A modem local to computer system 500 can receive the data on the telephone line and use an infra-red transmitter to convert the data to an infra-red signal. An infra-red detector can receive the data carried in the infra-red signal and appropriate circuitry can place the data on bus 502. Bus 502 carries the data to main memory 506, from which processor 504 retrieves and executes the instructions. The instructions received by main memory 506 may optionally be stored on storage device 510 either before or after execution by processor 504.

Computer system 500 also includes a communication interface 518 coupled to bus 502. Communication interface 518 provides a two-way data communication coupling to a network link 520 that is connected to a local network 522. For example, communication interface 518 may be an integrated services digital network (ISDN) card, cable modem, satellite modem, or a modem to provide a data communication connection to a corresponding type of telephone line. As another example, communication interface 518 may be a local area network (LAN) card to provide a data communication connection to a compatible LAN. Wireless links may also be implemented. In any such implementation, communication interface 518 sends and receives electrical, electromagnetic or optical signals that carry digital data streams representing various types of information.

Network link 520 typically provides data communication through one or more networks to other data devices. For example, network link 520 may provide a connection through local network 522 to a host computer 524 or to data equipment operated by an Internet Service Provider (ISP) 526. ISP 526 in turn provides data communication services through the worldwide packet data communication network now commonly referred to as the “Internet” 528. Local network 522 and Internet 528 both use electrical, electromagnetic or optical signals that carry digital data streams. The signals through the various networks and the signals on network link 520 and through communication interface 518, which carry the digital data to and from computer system 500, are example forms of transmission media.

Computer system 500 can send messages and receive data, including program code, through the network(s), network link 520 and communication interface 518. In the Internet example, a server 530 might transmit a requested code for an application program through Internet 528, ISP 526, local network 522 and communication interface 518.

The received code may be executed by processor 504 as it is received, and/or stored in storage device 510, or other non-volatile storage for later execution.

### 9. Miscellaneous; Extensions

Unless otherwise defined, all terms (including technical and scientific terms) are to be given their ordinary and customary meaning to a person of ordinary skill in the art, and are not to be limited to a special or customized meaning unless expressly so defined herein.

This application may include references to certain trademarks. Although the use of trademarks is permissible in patent applications, the proprietary nature of the marks should be respected, and every effort made to prevent their use in any manner which might adversely affect their validity as trademarks.

Embodiments are directed to a system with one or more devices that include a hardware processor and that are configured to perform any of the operations described herein and/or recited in any of the claims below.

In an embodiment, a computer program product includes instructions that, when executed by one or more hardware processors, causes performance of any of the operations described herein and/or recited in any of the claims.

In an embodiment, one or more non-transitory computer-readable storage media store instructions that, when executed by one or more hardware processors, cause performance of any of the operations described herein and/or recited in any of the claims. As used herein, the term “non-transitory computer-readable medium” refers to any tangible storage medium that stores computer-executable instructions for execution by one or more hardware processors in a computing device(s). The term “non-transitory” excludes transitory, propagating signals per se, such as carrier waves or other electromagnetic signals, but includes all forms of physical storage media.

In an embodiment, a method comprises operations described herein and/or recited in any of the claims, the method being executed by at least one device including a hardware processor.

Any combination of the features and functionalities described herein may be used in accordance with one or more embodiments. In the foregoing specification, embodiments have been described with reference to numerous specific details that may vary from implementation to implementation. The specification and drawings are, accordingly, to be regarded in an illustrative rather than a restrictive sense. The sole and exclusive indicator of the scope of the disclosure, and what is intended by the applicants to be the scope of the disclosure, is the literal and equivalent scope of the set of claims that issue from this application, in the specific form in which such claims issue, including any subsequent correction.

What is claimed is:

1. A method comprising:
accessing a plurality of clinical expressions, a first clinical expression of the plurality of clinical expressions comprising a first clinical concept of a plurality of clinical concepts, wherein a first clinical concept relationship, of a plurality of clinical concept relationships, associates the first clinical concept with a second clinical concept of the plurality of clinical concepts;
generating a first set of vector embeddings for the plurality of clinical expressions by applying a first vector embedding function to textual representation of each clinical expression of the plurality of clinical expressions;
generating a second set of vector embeddings for each clinical concept of the plurality of clinical concepts by applying a second vector embedding function to textual representation of each clinical concept of the plurality of clinical concepts;
generating a third set of vector embeddings for each of the plurality of clinical concept relationships associated with each clinical concept of the plurality of clinical concepts by applying a third vector embedding function to textual representation of each of the plurality of clinical concept relationships;
accessing a plurality of genomic expressions, a genomic expression, of the plurality of genomic expressions, comprises a genomic concept relationship between a pair of genomic concepts;
generating a fourth set of vector embeddings for the plurality of genomic expressions by applying a fourth vector embedding function to textual representation of each of the plurality of genomic expressions;
based on one or more of the first set of vector embeddings, the second set of vector embeddings, the third set of vector embeddings, or the fourth set of vector embeddings:
identifying a set of heterogenous relationships between genomic concepts of the plurality of genomic expressions and clinical concepts of the plurality of clinical concepts;
generating a fifth set of vector embeddings for the set of heterogenous relationships between genomic concepts and clinical concepts; and
using the first set of vector embeddings, the second set of vector embeddings, the third set of vector embeddings, the fourth set of vector embeddings and the fifth set of vector embeddings, training a multilevel classifier to identify one or more relationships of a patient condition to one or more genomic concepts and one or more clinical concepts to generate a trained multilevel classifier,
wherein the method is performed by at least one device including a hardware processor.

2. The method of claim 1, wherein generating the first set of vector embeddings comprises:
aggregating textual representations for each clinical concept associated with a first clinical expression to form a first set of aggregated text; and
applying the first vector embedding function to the first set of aggregated text.

3. The method of claim 1, wherein generating the second set of vector embeddings comprises:
aggregating textual representation of (i) a first clinical concept and an attribute associated with the first clinical concept to form a second set of aggregated text; and
applying the second vector embedding function to the second set of aggregated text.

4. The method of claim 1, wherein generating the third set of vector embeddings comprises:
aggregating textual representations of (i) a first clinical concept, (ii) a second clinical concept, and (iii) a type of relationship between the first clinical concept and the second clinical concept to form a third set of aggregated text; and
applying the third vector embedding function to the third set of aggregated text.

5. The method of claim 1, wherein generating the fourth set of vector embeddings comprises:
aggregating textual representations of (i) a first genomic concept, (ii) a second genomic concept, and (iii) a type of relationship between the first genomic concept and the second genomic concept to form a fourth set of aggregated text; and
applying a fourth vector embedding function to the fourth set of aggregated text.

6. The method of claim 1, wherein identifying the set of heterogenous relationships comprises:
generating a first similarity score between a first genomic concept and a first clinical concept;
determining that the first similarity score exceeds a threshold score; and
determining a type of relationship between the first genomic concept and the first clinical concept.

7. The method of claim 1, wherein generating the fifth set of vector embeddings for set of heterogenous relationships comprises:
aggregating textual representation of (i) a first genomic concept, (ii) a first clinical concept, and (iii) a type of relationship between the first genomic concept and the first clinical concept to form a fifth set of aggregated text; and
applying a fifth vector embedding function to the fifth set of aggregated text.

8. The method of claim 1, further comprising:
accessing patient data for a patient, the patient data comprising a first patient condition; and
applying the patient data to the trained multilevel classifier to identify one or more relationships of the first patient condition to one or more genomic concepts and one or more clinical concepts.

9. The method of claim 1, further comprising:
accessing patient data for a patient, the patient data comprising a first patient condition; and
applying the patient data to the trained multilevel classifier to identify one or more relationships of the first patient condition to one or more genomic concepts and one or more clinical concepts,
wherein the patient is treated in accordance with the one or more relationships identified of the first patient condition to the one or more genomic concepts and one or more clinical concepts.

10. The method of claim 1, wherein training the multilevel classifier comprises supervised and unsupervised training, wherein the multilevel classifier identifies previously unidentified relationships.

11. The method of claim 1, further comprising:
using a multi-output classifier to identify classifications for each vector embedding of the first set of vector embeddings, the second set of vector embeddings, the third set of vector embeddings, the fourth set of vector embeddings and the fifth set of vector embeddings,
wherein training the multilevel classifier further comprises providing the multilevel classifier with the classifications for each of the first set of vector embeddings, the second set of vector embeddings, the third set of vector embeddings, the fourth set of vector embeddings, and the fifth set of vector embeddings.

12. One or more non-transitory computer-readable media storing program instructions that, when executed by one or more hardware processors, cause performance of operations comprising:
accessing a plurality of clinical expressions, a first clinical expression of the plurality of clinical expressions comprising a first clinical concept of a plurality of clinical concepts, wherein a first clinical concept relationship, of a plurality of clinical concept relationships, associates the first clinical concept with a second clinical concept of the plurality of clinical concepts;
generating a first set of vector embeddings for the plurality of clinical expressions by applying a first vector embedding function to textual representation of each clinical expression of the plurality of clinical expressions;
generating a second set of vector embeddings for each clinical concept of the plurality of clinical concepts by applying a second vector embedding function to textual representation of each clinical concept of the plurality of clinical concepts;
generating a third set of vector embeddings for each of the plurality of clinical concept relationships associated with clinical concepts of the plurality of clinical concepts by applying a third vector embedding function to textual representation of each of the plurality of clinical concept relationships;
accessing a plurality of genomic expressions, a genomic expression, of the plurality of genomic expressions, comprises a genomic concept relationship between a pair of genomic concepts;
generating a fourth set of vector embeddings for the plurality of genomic expressions by applying a fourth vector embedding function to textual representation of each of the plurality of genomic expressions;
based on one or more of the first set of vector embeddings, the second set of vector embeddings, the third set of vector embeddings or the fourth set of vector embeddings:
identifying a set of heterogenous relationships between genomic concepts of the plurality of genomic expressions and the clinical concepts of the plurality of clinical concepts;
generating a fifth set of vector embeddings for the set of heterogenous relationships between genomic concepts and clinical concepts; and
using the first set of vector embeddings, the second set of vector embeddings, the third set of vector embeddings, the fourth set of vector embeddings and the fifth set of vector embeddings, training a multilevel classifier to identify one or more relationships of a patient condition to one or more genomic concepts and one or more clinical concepts to generate a trained multilevel classifier.

13. The one or more non-transitory computer-readable media of claim 12, wherein generating the first set of vector embeddings comprises:
aggregating textual representations for each clinical concept associated with a first clinical expression to form a first set of aggregated text;
applying the first vector embedding function to the first set of aggregated text,
wherein generating a second set of vector embeddings comprises:
aggregating textual representation of (i) a first clinical concept and an attribute associated with the first clinical concept to form a second set of aggregated text;
applying the second vector embedding function to the second set of aggregated text,
wherein generating the third set of vector embeddings comprises:
aggregating textual representations of (i) a first clinical concept, (ii) a second clinical concept, and (iii) a type of relationship between the first clinical concept and the second clinical concept to form a third set of aggregated text;
applying the third vector embedding function to the third set of aggregated text, wherein generating the fourth set of vector embeddings comprises:
aggregating textual representations of (i) a first genomic concept, (ii) a second genomic concept, and (iii) a type of relationship between the first genomic concept and the second genomic concept to form a fourth set of aggregated text; and
applying a fourth vector embedding function to the fourth set of aggregated text.

14. The one or more non-transitory computer-readable media of claim 12, wherein identifying the set of heterogenous relationships comprises:
generating a first similarity score between a first genomic concept and a first clinical concept;
determining that the first similarity score exceeds a threshold score; and
determining a type of relationship between the first genomic concept and the first clinical concept.

15. The one or more non-transitory computer-readable media of claim 12, wherein generating the fifth set of vector embeddings for the set of heterogenous relationships comprises:
aggregating textual representation of (i) a first genomic concept, (ii) a first clinical concept, and (iii) a type of relationship between the first genomic concept and the first clinical concept to form a fifth set of aggregated text; and
applying a fifth vector embedding function to the fifth set of aggregated text.

16. The one or more non-transitory computer-readable media of claim 12, wherein the operations further comprise:
accessing patient data for a patient, the patient data comprising a first patient condition; and
applying the patient data to the trained multilevel classifier to identify one or more relationships of the first patient condition to one or more genomic concepts and one or more clinical concepts.

17. The one or more non-transitory computer-readable media of claim 12, wherein the operations further comprise:
accessing patient data for a patient, the patient data comprising a first patient condition; and
applying the patient data to the trained multilevel classifier to identify one or more relationships of the first patient condition to one or more genomic concepts and one or more clinical concepts,
wherein the patient is treated in accordance with the one or more relationships identified of the first patient condition to the one or more genomic concepts and one or more clinical concepts.

18. The one or more non-transitory computer-readable media of claim 12, wherein training the multilevel classifier comprises supervised and unsupervised training, wherein the multilevel classifier identifies previously unidentified relationships.

19. The one or more non-transitory computer-readable media of claim 12, wherein the operations further comprise:
using a multi-output classifier to identify classifications for each vector embedding of the first set of vector embeddings, the second set of vector embeddings, the third set of vector embeddings, the fourth set of vector embeddings, and the fifth set of vector embeddings,
wherein training the multilevel classifier further comprises providing the multilevel classifier with the classifications for each of the first set of vector embeddings, the second set of vector embeddings, the third set of vector embeddings, the fourth set of vector embeddings, and the fifth set of vector embeddings.

20. A system comprising:
one or more hardware processors;
one or more non-transitory computer-readable media; and
program instructions stored on the one or more non-transitory computer-readable media that, when executed by the one or more hardware processors, cause the system to perform operations comprising:
accessing a plurality of clinical expressions, a first clinical expression of the plurality of clinical expressions comprising a first clinical concept of a plurality of clinical concepts, wherein a first clinical concept relationship, of a plurality of clinical concept relationships, associates the first clinical concept with a second clinical concept of the plurality of clinical concepts;
generating a first set of vector embeddings for the plurality of clinical expressions by applying a first vector embedding function to textual representation of each clinical expression of the plurality of clinical expressions;
generating a second set of vector embeddings for each clinical concept of the plurality of clinical concepts by applying a second vector embedding function to textual representation of each clinical concept of the plurality of clinical concepts;
generating a third set of vector embeddings for each of the plurality of clinical concept relationships associated with clinical concepts of the plurality of clinical concepts by applying a third vector embedding function to textual representation of each of the plurality of clinical concept relationships;
accessing a plurality of genomic expressions, a genomic expression, of the plurality of genomic expressions, comprises a genomic concept relationship between a pair of genomic concepts;
generating a fourth set of vector embeddings for the plurality of genomic expressions by applying a fourth vector embedding function to textual representation of each of the plurality of genomic expressions;
based on one or more of the first set of vector embeddings, the second set of vector embeddings, the third set of vector embeddings or the fourth set of vector embeddings:
identifying a set of heterogenous relationships between genomic concepts of the plurality of genomic expressions and the clinical concepts of the plurality of clinical concepts;
generating a fifth set of vector embeddings for the set of heterogenous relationships between genomic concepts and clinical concepts; and
using the first set of vector embeddings, the second set of vector embeddings, the third set of vector embeddings, the fourth set of vector embeddings, and the fifth set of vector embeddings, training a multilevel classifier to identify one or more relationships of a patient condition to one or more genomic concepts and one or more clinical concepts to generate a trained multilevel classifier.
